(19) United States
(12) Patent Application Publication
(10) Pub. No.: US 2014/0105829 A1
(43) Pub. Date: Apr. 17, 2014

(54) THERAPEUTIC NANOEMULSION FORMULATION FOR THE TARGETED DELIVERY OF DOCETAXEL AND METHODS OF MAKING AND USING THE SAME

(71) Applicant: Nemucore Medical Innovations, Inc., Worcester, MA (US)

(72) Inventors: Srinivas Ganta, Jamaica Plain, MA (US); Timothy P. Coleman, Wellesley, MA (US)

(73) Assignee: Nemucore Medical Innovations, Inc., Worcester, MA (US)

(21) Appl. No.: 14/053,754

(22) Filed: Oct. 15, 2013

Related U.S. Application Data
(60) Provisional application No. 61/713,690, filed on Oct. 15, 2012.

(51) Int. Cl. A61K 9/107; A61K 49/18; A61K 31/337

(52) U.S. Cl. CPC A61K 9/1075; A61K 31/337; A61K 49/1806
USPC 424/9.364; 514/449; 424/174.1; 424/9.3; 435/375

(57) ABSTRACT

A novel nanoemulsion formulation useful for the delivery of docetaxel chemotherapeutic agents is provided, as well as methods of their preparation and use in cancer patients and for cancer imaging.

### CROSS-REFERENCE TO RELATED APPLICATION

This application claims priority to and the benefit of U.S. provisional patent application No. 61/713,690, filed on Oct. 15, 2012, the entire content of which is hereby incorporated by reference herein.

### STATEMENT REGARDING FEDERALLY SPONSORED RESEARCH OR DEVELOPMENT

Part of the work leading to this invention was carried out with United States Government support provided under a grant from the National Institutes of Health, Grants Nos. R01CA158881 and U54CA151881. Therefore, the U.S. Government has certain rights in this invention.

### FIELD OF THE INVENTION

The present disclosure relates to medicine and pharmacology, and more particularly, to cancer therapy and diagnostics.

### BACKGROUND

Chemotherapeutic agents are widely used in cancer therapy. However, in most cases these treatments do not cure the disease. Challenges for effective therapy are the serious side-effects of many cancer drugs, insufficient concentration and short residence time of therapeutic agents at the site of disease, multi-drug resistance (MDR) and the hydrophobicity of pharmaceutical agents.

Hydrophobicity of novel or existing pharmaceutical agents limits the range of therapies for cancer treatment. Almost one third of the drugs in the United States Pharmacopeia (http://www.usp.org/) are hydrophobic and are either insoluble or poorly soluble (Savić et al. (2006) J. Drug Target. 14(6):343-55). Additionally, a majority of novel compounds being discovered are either insoluble or poorly soluble in water. As a result, many potential new chemical entities are being dropped in the early phases of development because of poor solubility in water.

With solubility as a major issue, some highly therapeutic drugs are often formulated with surfactants or co-solvents, which have associated toxic side effects, and stability, sterility, and mass commercial production issues have been common. Additionally, the existing delivery formulations for hydrophobic drugs have a limited ability to overcome the different transport barriers in biological systems.

Serious side-effects also occur when chemotherapeutic agents interact with normal tissue (Jain (2003) Nat. Med. 9(6):685-693). Lack of disease site specificity contributes to systemic toxicity as the therapeutic agent builds up in non-diseased tissues.

Numerous nanodelivery systems with and without site-specific binding moieties have been developed to address some of these problems. For example, a liposomal doxorubicin formulation (Doxil/Caelyx) increases drug concentration in tumors with concomitant decreased side effects. Other examples are poly(epsilon-caprolactone) (PCL) nanoparticles and poly(ethylene oxide)-polypropylene oxide) (PEO-PPO-PEO) triblock copolymer nanoparticles which have been used to improve the solubility and delivery of hydrophobic drugs (Chawla et al. (2003) AAPS Pharm. Sci. 5(1):28-34).

Another obstacle impeding cancer treatment, MDR, involves inherent or acquired resistance to chemotherapeutic agents. Many first line chemotherapeutic agents elicit a response, which causes tumor shrinkage, but often these tumors develop resistance to the chemotherapeutic agent before affecting a cure. For instance, chemotherapeutic taxane compounds (including docetaxel and paclitaxel) elicit a good initial response, but later, treated patients relapse because they develop resistance to the drug (Hopper-Borge et al. (2004) Cancer Res. 64(14):4927-4930).

Effective cancer therapy also suffers from the lack of early data on delivery of a particular therapeutic agent to tumors, and thus effectiveness is too slowly manifested. Patients often proceed with a course of treatment for an extended period of time, while suffering associated side effects and poor quality of life, only to find out that the particular treatment is not effective.

Accordingly, a need exists for therapeutic delivery systems, which can efficiently deliver therapeutic levels of drug to disease sites with fewer or no side effects and without resulting in MDR. There is also a need to expand the range of therapeutics that can be used for cancer treatment. In addition, a need also exists for imaging capabilities that will allow for quick determination as to whether therapy is accumulating in the disease site and whether a patient should proceed with a particular course of treatment.

### SUMMARY

It has been discovered that the formulation of a docetaxel compound into certain oil-containing nanoemulsions can increase the efficacy and efficiency of chemotherapeutic treatments. It has also been discovered that the use of certain nanoemulsion formulations having targeting ligands can overcome docetaxel MDR. Resistance is a natural cellular self-defense mechanism developed by evolution to protect cells from toxic natural products and other environmental stressors. By actively transporting docetaxel into the cell via endocytosis by a targeting ligand, the nanoemulsion formulation bypasses this defense mechanism, allowing for the delivery of chemotherapeutic docetaxel directly into the cancer cells of patients diagnosed as multidrug resistant. Therefore, the present disclosure provides a less toxic and more effective docetaxel chemotherapeutic to treat recurrent multidrug resistant cancers.

These discoveries have been exploited to develop the present disclosure, which, in one aspect, is a nanoemulsion formulation comprising an oil phase, an interfacial surface membrane, an aqueous phase, a docetaxel compound dispersed in the oil phase, and a targeting ligand comprising a folate receptor-targeting ligand.

In some embodiments, the oil phase of the nanoemulsion formulation comprises flax seed oil, hemp oil, pumpkin seed oil, wheat germ oil, rice bran oil, canola oil, or any combination thereof.

In certain embodiments, the interfacial surface membrane of the nanoemulsion formulation comprises an emulsifier, a stabilizer, or any combination of thereof. In particular embodiments, the emulsifier is egg lecithin, soy lecithin, phosphatidyl ethanolamine, phosphatidyl inositol, dimyristoylphosphatidyl choline, dimyristoylphosphatidyl ethyl-N-dimethyl propyl ammonium hydroxide, or any combination of thereof. In some embodiments, the stabilizer comprises a polyethylene glycol derivative, a phosphatide, a polyglycerol mono oleate, or any combination thereof. In certain embodiments, the polyethylene glycol (PEG) derivative comprises PEG₂₀₀₀DSPE, PEG₃₄₀₀DSPE, PEG₅₀₀₀DSPE, or any combination thereof.

In some embodiments, the docetaxel compound of the nanoemulsion formulation comprises docetaxel, lauroyl docetaxel, dilauroyl docetaxel, trilauroyl docetaxel, or any combination thereof.

In certain embodiments, the folate receptor-targeting ligand of the nanoemulsion formulation comprises DSPE-PEG(2000)-cysteine-folic acid, DSPE-PEG(3400)-cysteine folic acid, DSPE-PEG(5000)-cysteine folic acid, an anti-folate receptor immunoglobulin, or a folate receptor-binding fragment of the anti-folate receptor immunoglobulin, or any combination thereof.

In some embodiments, the nanoemulsion formulation further comprises an imaging agent. In certain embodiments the imaging agent comprises a magnetic resonance imaging contrasting moiety. In particular embodiments, the contrasting moiety comprises gadolinium, iron oxide, and/or manganese. In specific embodiments the gadolinium imaging agent comprises Gd-DTPA-PE, Gd-DOTA-PE, and/or Gd-PAP-DOTA.

In another aspect, the present disclosure provides a method of imaging a cancer. The method comprises contacting the cancer with an amount of the nanoemulsion formulation according to the specification, which is sufficient to image the cancer. In certain embodiments, the cancer is in a mammal and the nanoemulsion formulation is administered to the mammal in an amount sufficient to image the cancer.

In yet another aspect, the present disclosure provides a method of inhibiting or killing cancer cells, comprising contacting the cancer cells with an amount of a nanoemulsion formulation according to the specification that is toxic to, and/or which inhibits the growth of, the cancer cells. In some embodiments, the cancer cells are in a mammal and the nanoemulsion is administered to the mammal in a therapeutically effective amount.

### DESCRIPTION OF THE FIGURES

The foregoing and other objects of the present disclosure, the various features thereof, as well as the invention itself may be more fully understood from the following description, when read together with the accompanying drawings in which:

FIG. 1 is a diagrammatic representation of a generic nanoemulsion formulation of the present disclosure;

FIG. 2 is a schematic representation of one non-limiting mode of synthesizing a DSPE-PEG-Cys-folate nanoemulsion formulation according to the disclosure;

FIG. 3A is a representation of a transmission electronic micrograph (TEM) of a non-targeted blank nanoemulsion formulation;

FIG. 3B is a representation of a TEM of a folate receptor-targeted blank nanoemulsion formulation;

FIG. 3C is a representation of a TEM of a non-targeted nanoemulsion formulation according to the disclosure;

FIG. 3D is a representation of a TEM of a folate receptor-targeted nanoemulsion according to the disclosure;

FIG. 4A is a graphic representation of the dynamic light scattering (DLS) results of a non-targeted blank nanoemulsion formulation showing the size distribution of the particles;

FIG. 4B is a graphic representation of the DLS results of a folate receptor-targeted blank nanoemulsion formulation showing the size distribution of the particles;

FIG. 4C is a graphic representation of the DLS results of a non-targeted nanoemulsion formulation according to the disclosure showing the size distribution of the particles;

FIG. 4D is a graphic representation of the DLS results of a folate receptor-targeted nanoemulsion formulation according to the disclosure showing the size distribution of the particles;

FIG. 5 is a schematic representation of one non-limiting mode of synthesizing DSPE-PEG-Cys-FA;

FIG. 6 is a schematic representation of one non-limiting method of preparing Gd³⁺-DTPA-PE;

FIG. 7A is a graphic representation of the stability of non-targeted and folate receptor-targeted nanoemulsion formulations upon 90% dilution in plasma;

FIG. 7B is a graphic representation of the stability of non-targeted and folate receptor-targeted nanoemulsion formulations upon 90% dilution in 0.9% sodium chloride solution;

FIG. 7C is a graphic representation of the stability of non-targeted and folate receptor-targeted nanoemulsion formulations upon 90% dilution in 5% dextrose solution;

FIG. 7D is a graphic representation of the stability of non-targeted and folate receptor-targeted nanoemulsion formulations upon 90% dilution in phosphate buffered saline, pH 7.4 solution;

FIG. 8 is a graphic representation of docetaxel release from non-targeted and folate receptor-targeted nanoemulsion formulations;

FIG. 9A are representations of fluorescent images of SKOV3 cells at 5 minutes, 15 minutes, 30 minutes, and 60 minutes after treatment with fluorescently labeled, non-targeted nanoemulsion formulations;

FIG. 9B are representations of fluorescent images of SKOV3 cells at 5 minutes, 15 minutes, 30 minutes, and 60 minutes after treatment with fluorescently labeled, folate receptor-targeted nanoemulsion formulations of the present disclosure;

FIG. 10A is a graphic representation of caspase 3/7 activity in SKOV3 cells treated with non-targeted nanoemulsion formulations, or folate receptor-targeted nanoemulsion formulations over time, relative to control treatments (none or Taxotere);

FIG. 10B is a representation of caspase 3/7 activity on SKOV3_{TR} cells treated with non-targeted or folate receptor-targeted nanoemulsion formulations over time, relative to control treatments (none or Taxotere);

FIG. 11A is a graphic representation of body weights of Nu/Nu nude mice following treatment with the maximum tolerated dose determination for docetaxel (7.5 mg/kg) administered in non-targeted and folate-targeted nanoemulsion formulations of the present disclosure, relative to Taxotere and untreated control;

FIG. 11B is a graphic representation of body weights of Nu/Nu nude mice following treatment with the maximum tolerated dose determination for docetaxel (10 mg/kg) administered in non-targeted and folate receptor-targeted nanoemulsion formulations of the present disclosure, relative to Taxotere and untreated control;

FIG. 11C is a graphic representation of body weights of Nu/Nu nude mice following treatment with the maximum tolerated dose determination for docetaxel (16 mg/kg) administered in non-targeted and folate receptor-targeted nanoemulsion formulations of the present disclosure, relative to Taxotere and untreated control;

FIG. 12 is a graphic representation of the concentration of docetaxel in plasma over time after treatment with 1 mg/ml, 5 mg/ml, and 10 mg/kg docetaxel in the folate receptor-targeted nanoemulsion formulation administered intravenously to CD-1 mice;

FIG. 13 is a graphic representation of the concentration of docetaxel over time in plasma of Nu/Nu mice injected intravenously with 10 mg/kg of Taxotere, docetaxel in a non-targeted nanoemulsion according to the disclosure, or docetaxel in a folate receptor-targeted nanoemulsion formulation according to the disclosure;

FIG. 14 is a graphic representation of the results of a cytotoxicity assay using SKOV3 and SKOV3_{TR} ovarian cancer cells treated with non-targeted and folate receptor-targeted nanoemulsion formulations of the present disclosure, relative to docetaxel in DMSO;

FIG. 15 is a graphic representation of the therapeutic efficacy of a folate receptor-targeted nanoemulsion formulation according to the disclosure, relative to control treatments (none or Taxotere) as measured by the survival rate of treated animals in days post-treatment;

FIG. 16 is a graphic representation of liver enzyme (alanine amino transferase (ALT) and aspartate amino-transferase (AST)) levels in taxotere-treated, non-targeted nanoemulsion formulation-treated animals or folate receptor-targeted nanoemulsion formulation-treated animals, demonstrating the safety of a non-targeted nanoemulsion formulation, or a folate receptor-targeted nanoemulsion formulation, relative to Taxotere;

FIG. 17A is a representation of magnetic resonance images (MRI) over a period of 24 hours of full body scans of mice treated with a Gd³⁺-DTPA-PE, Magnevist™;

FIG. 17B is a representation of MRIs over a period of 24 hours of full body scans of mice treated with a Gd³⁺-DTPA-PE, non-targeted nanoemulsion formulation; and

FIG. 17C is a representation of MRIs over a period of 24 hours of full body scans of mice treated with a Gd³⁺-DTPA-PE, folate receptor-targeted nanoemulsion formulation.

### DETAILED DESCRIPTION

Throughout this application, various patents, patent applications, and publications are referenced. The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

### Definitions

For convenience, certain terms employed in the specification, examples, and appended claims are collected here. Unless defined otherwise, all technical and scientific terms used herein have the same meaning as commonly understood by one of ordinary skill in the art to which this invention belongs. The initial definition provided for a group or term herein applies to that group or term throughout the present specification individually or as part of another group, unless otherwise indicated.

The articles “a” and “an” are used herein to refer to one or to more than one (i.e., to at least one) of the grammatical object of the article. By way of example, “an element” means one element or more than one element.

The term “or” is used herein to mean, and is used interchangeably with, the term “and/or,” unless context clearly indicates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

The expression “at least one” is used herein to mean one or more and thus includes individual components as well as mixtures/combinations.

“Chemotherapeutic agent” as used herein means an agent that prevents or inhibits the development, growth or proliferation of malignant cells.

“Cancer” as used herein means the uncontrolled growth of abnormal cells.

“Stable docetaxel-containing formulation” as used herein means a formulation containing a docetaxel or docetaxel derivative wherein the derivative is stable for transformation for a time sufficient to be therapeutically useful. Exemplary stable docetaxel-containing formulations are the nanoemulsion formulations of the present disclosure, which comprise docetaxel compounds.

“Docetaxel compound” as used herein encompasses docetaxel and docetaxel derivatives, including salts having anticancer docetaxel activity. One nonlimiting docetaxel compound is Taxotere, which is docetaxel trihydrate dispersed in polysorbate 80, and an ethanol.

“Stabilizer” as used herein means an agent that prevents or slows the transformation or deactivation of a docetaxel-containing compound or ion in a docetaxel-containing nanoemulsion formulation.

“Patient” as used herein means a human or animal in need of treatment for cancer.

“Nanoemulsion formulation” as used herein means a novel nanoemulsion comprising an oil phase; an interfacial surface membrane; an aqueous phase; a docetaxel compound dispersed in the oil phase; and a targeting ligand comprising a folate receptor-targeting ligand.

“Nanoemulsion” as used herein means a colloidal dispersion comprised of omega-3, -6 or -9 fatty acid rich oils in an aqueous phase and thermo-dynamically stabilized by amphiphilic surfactants, which make up the interfacial surface membrane, produced using a high shear microfluidization process usually with droplet diameter within the range of about 80-220 nm.

“Oil phase” as used herein means the internal hydrophobic core of the nanoemulsion in which a docetaxel compound is dispersed and refers either to a single pure oil or a mixture of different oils present in the core. The oil phase is comprised of generally regarded as safe grade, parenterally injectable excipients generally selected from omega-3, omega-6 or omega-9 polyunsaturated unsaturated fatty acid (PUFA) or monounsaturated fatty acid rich oils.

“Aqueous phase” is comprised of isotonicity modifiers and pH adjusting agents in sterile water for injection and forms as an external phase of the nanoemulsion formulation in which the oil phase is dispersed.

“Amphiphilic molecule or amphiphilic compound” as used herein means any molecule of bipolar structure comprising at least one hydrophobic portion and at least one hydrophilic portion. The hydrophobic portion distributes into the oil phase and hydrophilic portion distributes into aqueous phase forming an interfacial surface membrane and has the property of reducing the surface tension of water (g<55 mN/m) and of reducing the interface tension between water and an oil phase. The synonyms of amphiphilic molecule are, for example, surfactant, surface-active agent and emulsifier.

“Amphiphilic” as used herein means a molecule with both a polar, hydrophilic portion and a non-polar, hydrophobic portion.

“Primary emulsifiers” as used herein means amphiphilic surfactants that constitute a major percentage of amphiphilic surfactants of the nanoemulsion formulation wherein they stabilize the formulation by forming an interfacial surface membrane around oil droplets dispersed in water, and further allow for surface modification with targeting ligands and imaging agents

“Co-emulsifiers” as used herein means amphiphilic surfactants used in conjunction with primary emulsifiers where they associate with the interfacial surface membrane, effectively lowering the interfacial tension between oil and water, and help in the formation of stable nanoemulsion formulations.

“Stabilizers” or “stealth agents” as used herein mean lipidated polyethylene glycols (PEG) where the lipid tail group distributes into the oil phase and hydrophilic PEG chains distribute into the aqueous phase of a nanoemulsion formulation, providing steric hindrance to mononuclear phagocytic system (MPS) cell uptake during the blood circulation, thus providing longer residence time in the blood and allowing for enhanced accumulation at tumor site through leaky tumor vasculature, a phenomenon termed as enhanced permeability and retention effect, largely present in wide variety of solid tumors. Other representative examples are a phosphatide, and a polyglycerol mono oleate,

“Targeting agents” as used herein encompass lipidated PEG bonded to tumor receptor-specific ligands at the end of PEG chains where the targeting ligand extends outside the oil droplets covered by an interfacial surface membrane into the aqueous phase. Targeting agents allow for interaction with tumor cells in vivo, forming a ligand-receptor complex, which is taken up by the tumor cells.

“Imaging agents” as used herein encompass metal ions (eg. gadolinium, iron and manganese), which provide contrast to visualize a disease site using magnetic resonance imaging (MRI), and the near infra-red fluorescent dyes (eg. DiR), which provide fluorescence at the disease site. These agents are either linked to lipidated chelates (eg. DTPA-PE and DOTA-PE) and incorporated in the nanoemulsion formulation at its interfacial surface membrane or are loaded inside the oil core of the nanoemulsion formulation according to the disclosure.

“Isotonicity modifiers” as used herein means agents that provide an osmolality (285-310 mOsm/kg) to the nanoemulsion formulation, thus maintaining isotonicity for parenteral injection.

“pH modifiers” as used herein means buffering agents that adjust the pH of nanoemulsion formulation to a value of about pH 6-7.4, thus preventing the hydrolysis of phospholipids upon storage.

“Preservatives” as used herein means antimicrobial agents that when added to the nanoemulsion formulation at about 0.001-005% w/v prevent bacterial growth during the storage of nanoemulsion formulation.

“Antioxidants” as used herein means agents that stop oxidation of oils comprised of fatty acids, thus preventing rancidification of oil phase and destabilization of the nanoemulsion formulation.

### 1. Nanoemulsion Formulations

The present disclosure provides novel nanoemulsion formulations useful for cancer treatment and imaging of tumors and cancer cells. This formulation comprises an oil phase, an interfacial surface membrane, an aqueous phase, a chemotherapeutic agent comprising a docetaxel compound dispersed in the oil phase, and a targeting ligand comprising a folate receptor-targeting ligand.

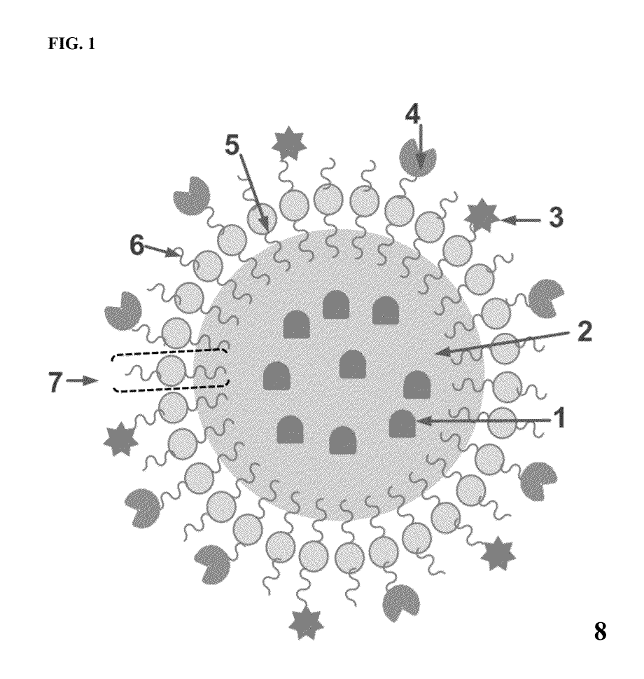

FIG. 1 is a non-limiting schematic representation of a nanoemulsion formulation of the present disclosure. In this figure, 1 represents a chemotherapeutic agent comprising a docetaxel compound dispersed in the oil phase 2. 2 is encapsulated within the interfacial surface membrane 7 which comprise emulsifiers 5 and stabilizers 6. The polar, hydrophilic portion of these amphiphiles of the interfacial surface membrane projects into the aqueous phase 8, and the non-polar, hydrophobic portion of these amphiphiles projects into the oil phase 2. Some of the stabilizers 6 in the interfacial surface membrane are bonded to targeting ligand 4. 3 represents an imaging agent attached to emulsifier 5 in the interfacial surface membrane 7.

The nanoemulsion formulation of the present disclosure may also comprise a co-emulsifier, a preservative, an antioxidant, a pH adjusting agent, an isotonicity modifier, or any combination thereof.

Various non-limiting examples of the components of the nanoemulsion formulation of the present disclosure and their corresponding proportions are provided in Table I and discussed below.

TABLE I
Component Proportions for Representative Formulations

| Category | Examples | % w/w |
|---|---|---|
| Oil Phase | Flax seed oil (omega-3 and omega-6 | 5-30 |
| | polyunsaturated components), | |
| | Hemp oil, | |
| | wheat germ, | |
| | rice bran, | |
| | canola, | |
| | pumpkin seed oil | |
| Primary Emulsifiers | egg lecithin, | 0.5-5.0 |
| | soy lecithin | |
| Co-Emulsifiers | phosphatidyl ethanolamine, | 0.1-1.0 |
| | phosphatidyl inositol, | |
| | dimyristoylphosphatidyl choline, | |
| | dimyristoylphosphatidyl | |
| | ethyl-N-dimethyl propyl ammonium | |
| | hydroxide | |
| Stealth Agents | PEGylated lipids | 0.1-0.75 |

### A. Docetaxel Compounds

The novel nanoemulsion formulations according to the disclosure have improved delivery and greatly reduced patient toxicity. They include at least one encapsulated docetaxel compound that can be associated with the interfacial membrane surface of, encapsulated within and surrounded by, and/or dispersed throughout the oil core.

Docetaxel has the following chemical structure:

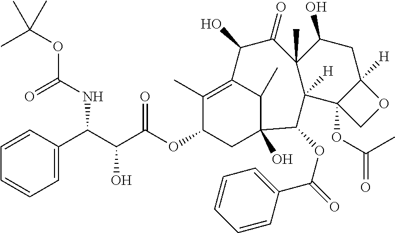

The docetaxel compounds include docetaxel and its derivatives and salts such as, but not limited to, lauroyl docetaxel, dilauroyl docetaxel, trilauroyl docetaxel, and any combination thereof, which are active in treating cancer.

A docetaxel compound is an anti-mitotic chemotherapeutic agent that interferes with cell division (see, e.g., Lyseng-Williamson et al. (2005) Drugs 65(17):2513-31; Clarke et al. (1999) Clin. Pharmacokinet 36(2):99-114; Michael et al. (2009) Prostate Can. & Prostatic Dis. 12 (1):13-16). The cytotoxic activity of docetaxel compounds is understood to be exerted by promoting and stabilizing microtubule assembly, while preventing physiological microtubule disassembly in the absence of guanosine triphosphate (GTP). This activity leads to a significant decrease in free tubulin, which is needed for microtubule formation, and results in inhibition of mitotic cell division between metaphase and anaphase, preventing further cancer cell progeny. Because microtubules do not disassemble in the presence of docetaxel compounds, they accumulate inside the cell and initiate apoptosis. Apoptosis is also encouraged by the blocking of apoptosis-blocking bcl-2 oncoprotein.

Docetaxel compounds are effective against a wide range of known cancers including breast, colorectal, lung, ovarian, gastric, renal, and prostate cancer. Docetaxel compounds cooperate with other anti-neoplastic agents, and have greater cytotoxicity than paclitaxel. It has been discovered that the encapsulation of docetaxel compounds in the nanoemulsion formulations of the present disclosure aids in mitigating undesirable side-effects known to sometimes accompany their use.

### B. Oil Phase

One essential component of the nanoemulsion formulation according to the present disclosure is an oil phase comprising individual oil droplets and which represents the internal hydrophobic or oil core. The oil phase may be a single entity or a mixture. The average size of the oil droplets in the oil phase ranges from about 5 nm to 500 nm.

A wide variety of oils and methods for forming nanoemulsion formulations therefrom are known in the art of drug delivery. The oil phase of the disclosed nanoemulsion formulations may include at least one PUFA-rich oil, for example, a first oil that may contain polyunsaturated oil, for example linolenic acid, and optionally an oil that may be for example a saturated fatty acid, for example icosanaic acid.

Any oil can be used in accordance with the present invention. Oils can be natural or unnatural (synthetic) oils. Oils can be homogeneous or oils comprising two or more monounsaturated fatty acid or PUFA-rich oils. Contemplated oils may be biocompatible and/or biodegradable.

Biocompatible oils do not typically induce an adverse response (such as an immune response with significant inflammation and/or acute rejection) when inserted or injected into a living subject. Accordingly, the therapeutic nanoemulsion formulations contemplated herein can be non-immunogenic. The term “non-immunogenic” as used herein refers to endogenous growth factor in its native state which normally elicits no, or only minimal levels of, circulating antibodies, T-cells, or reactive immune cells, and which normally does not elicit in the individual an immune response against itself.

Biocompatibility typically refers to the lack of acute rejection of a material by at least a portion of the immune system. A non-biocompatible material implanted into a subject provokes an immune response that can be severe enough to cause rejection of the material by the immune system that cannot be adequately controlled, and often is of a degree such that the material must be removed from the subject. One simple test to determine biocompatibility is to expose a nanoemulsion formulation to cells in vitro. Biocompatible oils in the nanoemulsion formulation typically will not result in significant cell death at moderate concentrations, e.g., 50 μg/10⁶ cells. For instance, these biocompatible oils can cause less than about 20% cell death when exposed to or taken up by, fibroblasts or epithelial cells. Non-limiting examples of biocompatible oil useful in nanoemulsion formulations of the present disclosure include alpha linolenic acid, pinolenic acid, gamma linolenic, linoleic acid, oleic acid, icosenoic acid, palmitic acid, stearic acid, icosanaic acid, and derivatives thereof.

The contemplated biocompatible oils may be biodegradable, i.e., able to degrade, chemically and/or biologically, within a physiological environment, such as within the body. As used herein, “biodegradable” oils are those that, when introduced into cells, are broken down by the cellular machinery (biologically degradable) and/or by a chemical process, such as hydrolysis, (chemically degradable) into components that the cells can either reuse or dispose of without significant toxic effect on the cells. Both the biodegradable oils and their degradation byproducts can be biocompatible.

A useful oil is flax seed oil which is a biocompatible and biodegradable oil of alpha linolenic, linoleic, and oleic. Useful forms of this oil can be characterized by the ratio of alpha linolenic:linoleic:oleic. The degradation rate of flax seed oil can be adjusted by altering the alpha linolenic:linoleic:oleic ratio, e.g., having a molar ratio of about 65:5:30, about 65:20:15, about 55:15:30, or about 55:20:25.

The disclosed nanoemulsion formulations include an oil phase of saturated fatty acid, monounsaturated fatty acid or PUFA rich oils that are biocompatible and/or biodegradable.

Oil compositions suitable for use as the oil phase of the nanoemulsion formulations according to the present disclosure can be from any source rich in mono-saturated or polyunsaturated fatty acids, such as plant or animal sources. Chemically or enzymatically derivatized, or completely synthetic, monounsaturated or polyunsaturated fatty acids are included within the scope of suitable components for the oil phase of the nanoemulsion formulations of the present disclosure. The concentration of the mono-unsaturated or polyunsaturated fatty acid in the oil phase can range from about 2% to 100% (w/w), from about 5% to 100% (w/w), or greater than 10% from about 20%-80% (w/w). The concentration of the oil phase, in the nanoemulsion formulation can vary from about 5% w/w to 40% w/w, or from about 5% to 30% w/w. The concentration of hydrophobic chemotherapeutic agent soluble in the oil phase can range from 0.01% to 90% (w/w), from about 0.1% to 45% (w/w) or greater than 0.5%, from about 1%-30% (w/w). For example, the oils may contain high concentrations of mono-saturated or polyunsaturated fatty acids such as a concentration of greater than or equal to 10% (w/w) of at least one mono-unsaturated or polyunsaturated fatty acid of the omega-3, omega-6 or omega-9 family. A useful oil is one that can solubilize high concentrations of a hydrophobic chemotherapeutic agent including a docetaxel compound. For example, useful oils are those containing high concentrations of linolenic or linoleic acid, e.g., oils of flax seed oil, black currant oil, pine nut oil or borage oil, and fungal oils such as spirulina and the like, alone or in combination.

### C. Aqueous Phase

The aqueous phase of the nanoemulsion formulations according to the disclosure is purified and/or ultrapure water. This aqueous phase can contain isotonicity modifiers such as, but not limited to, glycerine, low molecular weight polyethylene glycol (PEG), sorbitol, xylitol, or dextrose. The aqueous phase can alternatively or also contain pH adjusting agents such as, but not limited to, sodium hydroxide, hydrochloric acid, free fatty acids (oleic acid, linoleic acid, stearic acid, palmitic acid) and their sodium and potassium salts, preservative parabens, such as, but not limited to, methyl paraben or propyl paraben; antioxidants such as, but not limited to, ascorbic acid, α-tocopherol, and/or butylated hydroxy anisole. The concentration of the aqueous phase in the present nanoemulsion formulations can vary from 30% to 90% (w/w).

### D. Interfacial Surface Membrane

(1) Emulsifiers

As described above, the interfacial surface may comprise an emulsifier and/or a stabilizer (stealth agent).

The emulsifiers form part of the interface between the hydrophobic or oil core and the aqueous phase. The term “interfacial surface membrane” as used herein applies to the interface of the oil and aqueous phase and may refer either to a single pure emulsifier or a mixture of different emulsifiers and/or a mixture of emulsifiers and other components, such as stealth agents present in the interfacial surface membrane of the nanoemulsion formulation. The interfacial surface membrane or corona can comprise degradable lipids or emulsifiers bearing neutral, cationic and/or anionic side chains. The average surface area of the interfacial surface membrane corona on the nanoemulsion formulations described herein from may range from 30,000 nm² to 600,000 nm².

The emulsifiers comprise individual amphiphilic lipids and/or amphiphilic polymers. At least one emulsifier is present at the interface between the oil phase and the aqueous phase. The emulsifier can be an amphiphilic molecule such as a nonionic and ionic amphiphilic molecules. For example, the emulsifier can consist of neutral, positively-charged, or negatively-charged, natural or synthetic phospholipids molecules such as, but not limited to, natural phospholipids including soybean lecithin, egg lecithin, phosphatidylglycerol, phosphatidylinositol, phosphatidylethanolamine, phosphatidic acid, sphingomyelin, diphosphatidylglycerol, phosphatidylserine, phosphatidylcholine and cardiolipin; synthetic phospholipids including dimyristoylphosphatidylcholine, dimyristoylphosphatidyl ethyl-N-dimethyl propyl ammonium hydroxide, dimyristoylphosphatidylglycerol, distearoylphosphatidylglycerol and dipalmitoylphosphatidylcholine; and hydrogenated or partially hydrogenated lecithins and phospholipids, e.g., from a natural source are used. The concentration of amphiphilic lipid in the nanoemulsion formulations can vary from about 0.5% to 15% (w/v), or from about 1% to 10% (w/v).

One nonlimiting example of a nanoemulsion formulation of the present disclosure comprises 1) an oil and 2) amphiphilic compounds of the interfacial surface membrane that surround or are dispersed within the oil and which form a continuous or discontinuous monomolecular layer. The interfacial surface membrane lowers the interfacial tension between the oil and aqueous phases, thereby enhancing the stability of the dispersed oil droplets in the surrounding aqueous phase. Further, the interfacial surface membrane of the nanoemulsion formulation localizes drugs, thereby providing therapeutic advantages by releasing the encapsulated chemotherapeutic drug, such as a docetaxel compound, at predetermined, appropriate times.

Often, an amphiphilic compound of the interfacial surface membrane has a polar head attached to a long hydrophobic tail. The polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. Exemplary amphiphilic compounds include, for example, one or a plurality of the following: naturally derived lipids, surfactants, or synthesized compounds with both hydrophilic and hydrophobic moieties.

Specific examples of amphiphilic compounds making up a representative emulsifier include phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of about 0.5%-2.5% (weight lipid/w oil), about between 1.0%-1.5% (weight lipid/w oil). Phospholipids, which may be used, include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

An amphiphilic compound of the interfacial membrane may include lecithin or phosphatidylcholine.

(2) Stabilizers

The stabilizer or stealth agent may or may not be part of the interfacial surface membrane. If it is a part of this membrane, it can be added with the emulsifier when preparing a nanoemulsion formulation of the present disclosure. One useful representative stabilizer is a PEGylated lipid. Some useful phospholipid molecules are natural phospholipids including polyethylene glycol (PEG) repeat units, which can also be referred to as a “PEGylated” lipid or lipidated PEG. Such PEGylated lipids can control inflammation and/or immunogenicity (i.e., the ability to provoke an immune response) and/or lower the rate of clearance from the circulatory system via the reticuloendothelial system (RES) and/or the mononuclear phagocyte system (MPS), due to the presence of the poly(ethylene glycol) groups, PEGylated soybean lecithin, PEGylated egg lecithin, PEGylated phosphatidylglycerol, PEGylated phosphatidylinositol, PEGylated phosphatidylethanolamine, PEGylated phosphatidic acid, PEGylated sphingomyelin, PEGylated diphosphatidylglycerol, PEGylated phosphatidylserine, PEGylated phosphatidylcholine and PEGylated cardiolipin; synthetic phospholipids including PEGylated dimyristoylphosphatidylcholine, PEGylated dimyristoylphosphatidylglycerol, PEGylated distearoylphosphatidylglycerol and PEGylated dipalmitoylphosphatidylcholine; and hydrogenated or partially hydrogenated PEGylated lecithins and PEGylated phospholipids. Such amphiphilic PEGylated lipids can be used alone or in combination. The concentration of amphiphilic PEGylated lipid in the nanoemulsions can vary from about 0.01% to 15% (w/v), or from about 0.05% to 10% (w/v).

Exemplary lipids that can be part of the PEGylated lipid include, but are not limited to, fatty acids such as long chain (e.g., C8-050), substituted, or unsubstituted hydrocarbons. A fatty acid group can be a C10-C20 fatty acid or salt thereof, a C15-C20 fatty acid or salt thereof, or a fatty acid can be unsaturated, monounsaturated, or polyunsaturated. For example, a fatty acid group can be one or more of butyric, caproic, caprylic, capric, lauric, myristic, palmitic, stearic, arachidic, behenic, lignoceric, palmitoleic, oleic, vaccenic, linoleic, alpha-linolenic, gamma-linoleic, arachidonic, gadoleic, arachidonic, eicosapentaenoic, docosahexaenoic, or erucic acid.

Other exemplary stabilizers are phosphatide, a polyglycerol mono oleate, PEG₂₀₀₀DSPE, PEG₃₄₀₀DSPE, PEG₅₀₀₀DSPE, or any combination thereof. Useful stabilizers are a PEG derivative, a phosphatide, and/or polyglycerol mono oleate and useful non-limiting PEG derivatives are PEG₂₀₀₀DSPE, PEG₃₄₀₀DSPE, PEG₅₀₀₀DSPE.

The PEGylation density may be varied as necessary to facilitate long-circulation in the blood (Perry et al. (2012) Nano Lett. 12:5304-5310). In some cases, the addition of PEG repeat units may increase plasma half-life of the nanoemulsion formulation, for instance, by decreasing the uptake of the nanoemulsion formulation by the MPS, while decreasing transfection/uptake efficiency by cells. Those of ordinary skill in the art will know of methods and techniques for PEGylating a lipid, for example, by using EDC (1-ethyl-3-(3-dimethylaminopropyl) carbodiimide hydrochloride) and NHS (N-hydroxysuccinimide) to react a lipid that will be in the corona of the nanoemulsion formulation to a PEG group terminating in an amine, by ring opening polymerization techniques (ROMP), or the like.

It is contemplated that PEG may include a terminal end group, for example, when PEG is not conjugated to a ligand. For example, PEG may terminate in a hydroxyl, a methoxy or other alkoxyl group, a methyl or other alkyl group, an aryl group, a carboxylic acid, an amine, an amide, an acetyl group, a guanidino group, or an imidazole. Other contemplated end groups include azide, alkyne, maleimide, aldehyde, hydrazide, hydroxylamine, alkoxyamine, or thiol moieties.

The interfacial surface membrane of the nanoemulsion formulations disclosed herein may alternatively contain poly(ester-ether)s. For example, the interfacial membrane surfaces of the nanoemulsion formulation can have repeat units joined by ester bonds (e.g., R—C(O)—O—R′ bonds) and ether bonds (e.g., R—O—R′ bonds). A biodegradable component of the interfacial membrane surface of the nanoemulsion formulation, such as a hydrolyzable biopolymer containing carboxylic acid groups, may be conjugated with poly(ethylene glycol) repeat units to form a poly(ester-ether) coating on the interfacial membrane surface of the nanoemulsion formulation.

The molecular weight of the PEG on interfacial membrane surface of the nanoemulsion formulation can be optimized for effective treatment as disclosed herein. For example, the molecular weight of a PEG may influence particle degradation rate (such as adjusting the molecular weight of a biodegradable PEG), solubility, water uptake, and drug release kinetics. For example, the molecular weight of the PEG can be adjusted such that the particle biodegrades in the subject being treated within a period of time ranging from a few hours, to 1 to 2 weeks, to 3 to 4 weeks, to 5 to 6 weeks, to 7 to 8 weeks, etc. One useful nanoemulsion formulation comprises a copolymer PEG conjugated to a lipid, the PEG having a molecular weight of about 1 kD to 20 kD, about 5 kD to 20 kD, or about 10 kD to 20 kD, and the lipid can have a molecular weight of about 200 Da to 3 kD, about 500 Da to 2.5 kD, or about 700 Da to 1.5 kD. An exemplary nanoemulsion formulation includes about 5 weight percent to about 30 weight percent monounsaturated or polyunsaturated fatty acid rich oil, or about 0.5 weight percent to about 5 weight percent primary emulsifier, or about 0.1 weight percent to about 1.0 weight percent co-emulsifiers or about 0.1 to about 0.75 weight percent, PEG-derivatives. Exemplary lipid-PEG copolymers can include a number average molecular weight of about 1.5 kD to about 25,000 kDa, or about 2 kD to about 20 kD.

The ratio of oil to emulsifier to stabilizer in the nanoemulsion formulation for example, flax seed oil to emulsifier to PEGylated lipid stabilizer, may be selected to optimize certain parameters such as size, chemotherapeutic agent release, and/or nanoemulsion formulation degradation kinetics.

### E. Folate Receptor-Targeting Ligands

The nanoemulsion formulations of the present disclosure may further comprise a folate receptor-targeting ligand, which is specific for a folate receptor on the cancer cells to be treated or imaged. When the nanoemulsion formulation approaches a cell having this receptor, it binds to it, thereby enabling endocytotic delivery of the chemotherapeutic docetaxel compound to the cell. In this way the nanoemulsion formulation may be delivered more accurately to the targeted cells and can overcome docataxel MDR.

The folate receptor (FR) has three isoforms: α, β, and γ. FR-α is a 38 kD glycosyl-phosphatidylinositol-anchored glycoprotein that binds folic acid (and internalizes it) with a kD of less than 1 nM and is highly expressed in a number of human tumors including ovarian (>85%), lung (>75%), breast (>60%) renal cell (>65%), brain, head, and neck (Fisher et al. (2008) J. Nucl. Med. 49:899-906). In normal tissue folate receptor expression is lower and limited to kidney tubuli, lung epithelium in the apical cell, the choroid plexus, and placenta. FR-α over expression is negatively associated with overall survival in ovarian and other cancers.

Useful folate receptor-targeting ligands include, but are not limited to, 1,2-distearoyl-sn-glycero-3-phosphoethanolamine-N-[methoxy(polyethylene glycol)-2000]-cysteine-folic acid (DSPE-PEG-cysteine-folic acid), 1,2-distearoyl-sn-glycero-3-phosphoethanolamine-N-[methoxy-(polyethylene glycol)-3400]-folic acid (EC119-PEG-DSPE), 1,2-distearoyl-sn-glycero-3-phosphoethanolamine-N-[folate(polyethylene glycol)-2000] (ammonium salt) (DSPE-PEG(2000) Folate), 1,2-distearoyl-sn-glycero-3-phosphoethanolamine-N-[folate (polyethylene glycol)-5000] (ammonium salt) (DSPE-PEG(5000) Folate) (Avanti Polar Lipids, Inc. Alabaster, Alabama), or other folate-targeting ligands comprising folate-PEG-ligand, an anti-folate receptor immunoglobulin, or a folate receptor-binding fragment thereof, and any combination thereof.

In some nanoemulsion formulations, the targeting moieties are attached, e.g., covalently bonded, to a lipid component of the nanoemulsion formulation. One exemplary nanoemulsion formulation comprising a docetaxel compound, an oil core comprising functionalized and non-functionalized oils, an interfacial surface membrane or corona, and a low-molecular weight targeting ligand, wherein the targeting ligand is covalently bonded, to the lipid component of the nanoemulsion formulation's interfacial surface membrane.

### F. Imaging Moieties

Nanoemulsion formulations of the present disclosure can further include imaging or contrast agents. The use of such agents allows physicians to track in real time the amount of chemotherapeutic agent actually reaching the site of disease. Physicians can then quickly decide whether a particular patient should continue with treatment. Useful imaging agents include, but are not limited to, paramagnetic agents such as gadolinium (Gd), iron oxide, iron platinum, and manganese. Useful Gd derivatives include 1,2-dimyristoyl-sn-glycero-3-phosphoethanolamine-N-diethylenetri-aminepentaacetic acid (Gd-DTPA-PE), 1,2-dimyristoyl-sn-glycero-3-phospho-ethanolamine-N-1,4,7,10-tetraazacyclododecane-1,4,7,10-tetraacetic acid (Gd-DOTA-PE), and 1,2-dimyristoyl-sn-glycero-3-paraazoxyphenetole-N-1,4,7,10-tetraazacyclododecane-1,4,7,10-tetraacetic acid (Gd-PAP-DOTA) (Avanti Polar Lipids, Inc. Alabaster, Alabama) or any combination thereof. These gadolinium-based MRI contrast moieties can be prepared or obtained and incorporated into a nanoemulsion formulation as described herein. Suitable imaging agents are gadolinium, iron oxide, iron, platinum, and manganese. Examples of suitable gadolinium imaging agents are Gd-DTPA-PE, Gd-DOTA-PE, Gd-PAP-DOTA.

Accordingly, a representative nanoemulsion formulation comprises an imaging moiety attached, e.g., covalently bonded, to a lipid component of the nanoemulsion formulation. One exemplary nanoemulsion formulation comprises a therapeutic docetaxel compound, an oil core comprising functionalized and non-functionalized oils, an interfacial surface membrane or corona, a folate receptor targeting ligand, and an imaging agent, wherein the imaging agent is covalently bonded to the lipid component of the nanoemulsion formulation's interfacial surface membrane.

In another exemplary nanoemulsion formulation, imaging moieties are soluble in the oil phase. For example, a nanoemulsion formulation comprises a chemotherapeutic docetaxel compound, an oil phase comprising functionalized and non-functionalized oils, an interfacial surface membrane, a folate receptor targeting ligand, and an imaging agent, wherein the imaging agent is soluble in the oil phase.

### 2. Preparation of Nanoemulsion Formulations

The nanoemulsion formulations of the present disclosure can be prepared from various intermediates and component constituents, for example, as described in Examples 1-4 below, and can be made using a microfluidizer (Microfluidics Corp., Newton, Mass.).

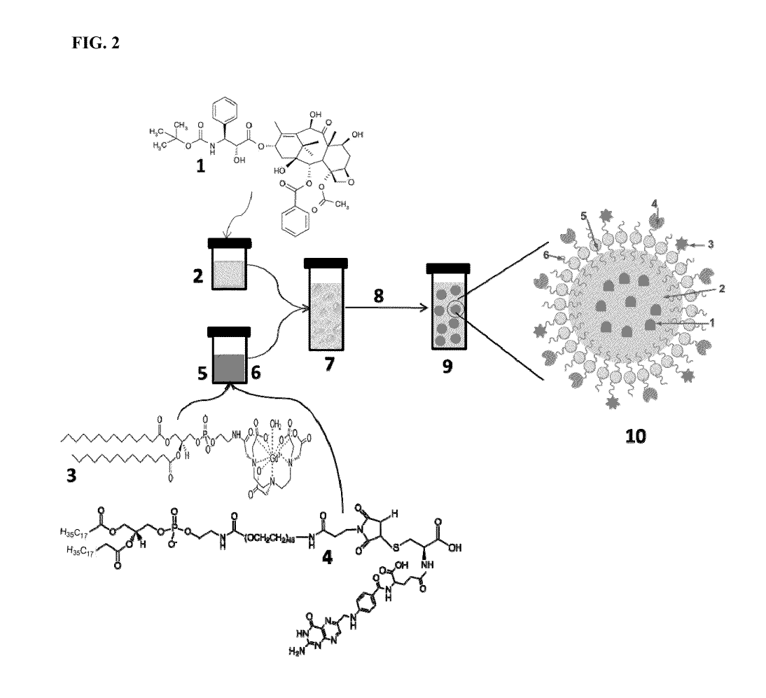

FIG. 2 shows a representative synthesis for one non-limiting, folate receptor-targeted, Gd-labeled nanoemulsion formulation of the present disclosure. In this figure, 1 is the active pharmaceutical ingredient docetaxel. 2 represents the compound of 1 being dissolved in chloroform and added to flax seed oil. Chloroform is removed using nitrogen, resulting in oil phase formation. 3 is the imaging moiety Gd-DTPA-PE. 4 is the targeting ligand Folate-cys-PEG-DSPE. 5 represents the compounds of 3 and 4 being added to egg lecithin and PEG₂₀₀₀DSPE in a glycerol water solution, resulting in aqueous phase formation. 6 represents the oil phase of 2 and the aqueous phase in 5 being combined to form the mixture used to form the coarse emulsion 7. 7 represents both the oil phase of 2 and aqueous phase of 5 being heated to 60° C. for 2 minutes and mixed to form coarse emulsion. 8 represents the coarse emulsion of 7 being emulsified using a high pressure homogenizer (LV1 Microfluidizer, Microfluidics, Newton, Mass.) at 25,000 psi for 10 cycles to obtain nanoemulsion formulation droplets of a size below 150 nm. 9 is the resulting nanoemulsion formulation of one possible embodiment of the present disclosure with a size below 200 nm. 10 is a representative drawing of an individual resulting nanoemulsion formulation.

### 3. Characterization of Nanoemulsion Formulations

The nanoemulsion formulations of the present disclosure may have a substantially spherical shape. For instance, the nanoemulsion formulations generally appear to be spherical, or non-spherical configuration, but upon shrinkage, may adopt a non-spherical configuration. These nanoemulsion formulations may have a characteristic dimension of less than about 1 μm, where the characteristic dimension of a nanoemulsion formulation is the diameter of a perfect sphere having the same volume as the nanoemulsion formulation. For example, the characteristic dimensions of the nanoemulsion formulation can be less than about 300 nm, less than about 200 nm, less than about 150 nm, less than about 100 nm, or less than about 50 nm in some cases. Some disclosed nanoemulsion formulations may have a diameter of about 50 nm to 200 nm, or about 50 nm to180 nm, about 80 nm to 160 nm, or about 80 nm to 150 nm.

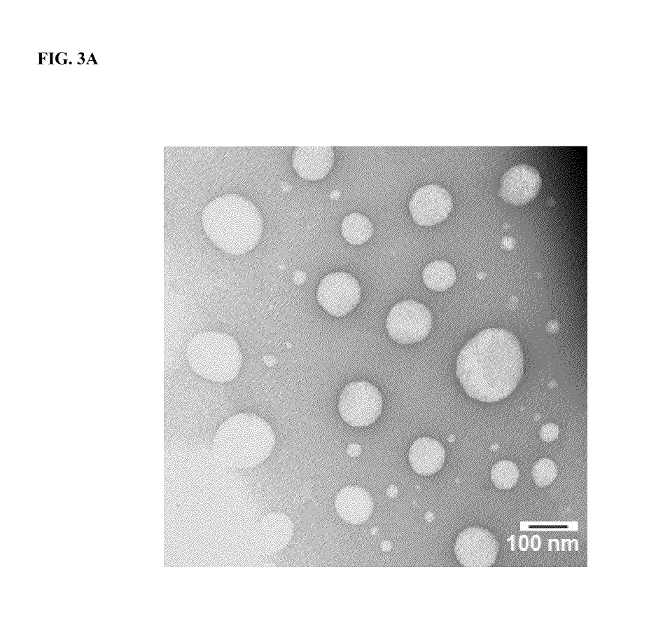

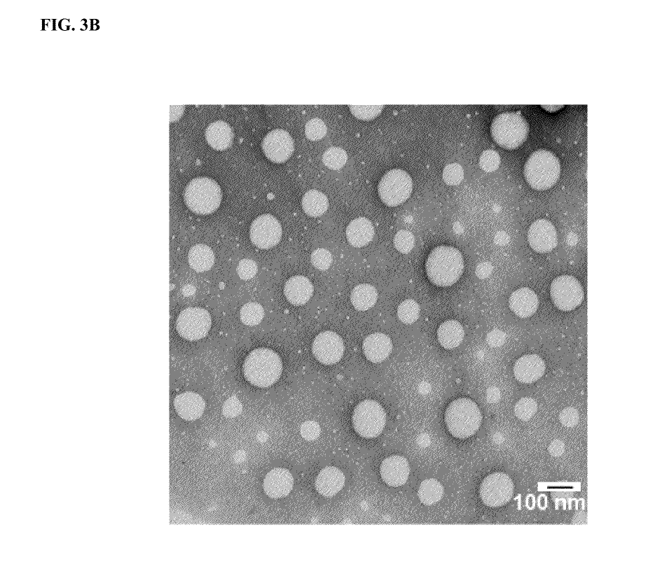

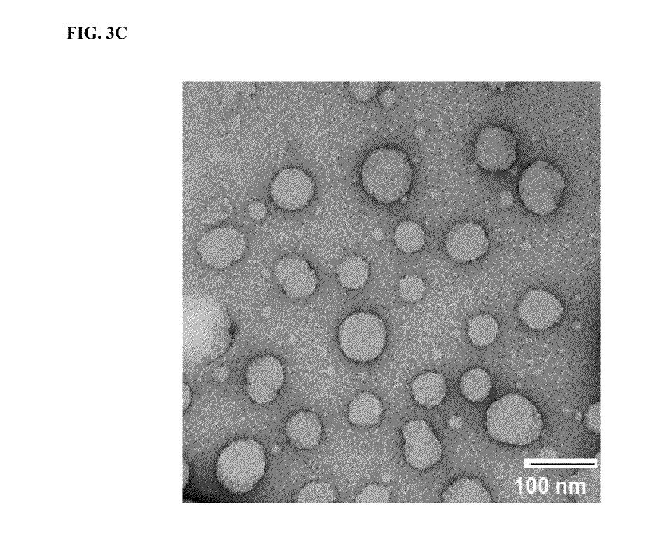

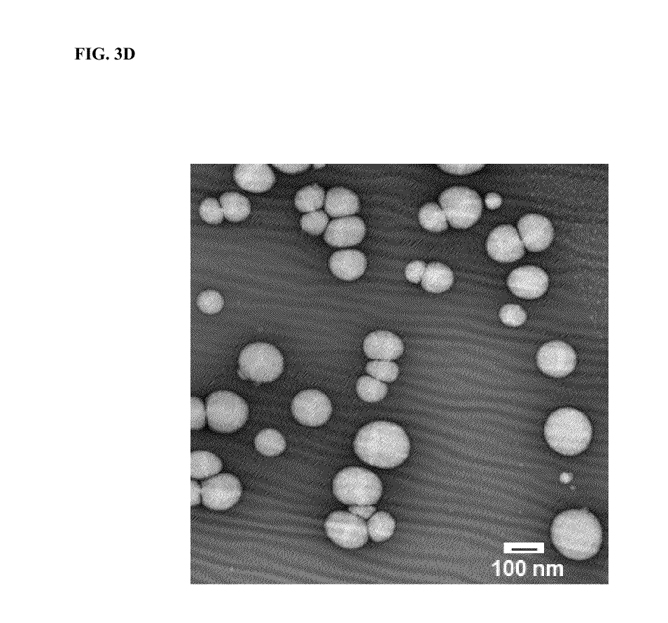

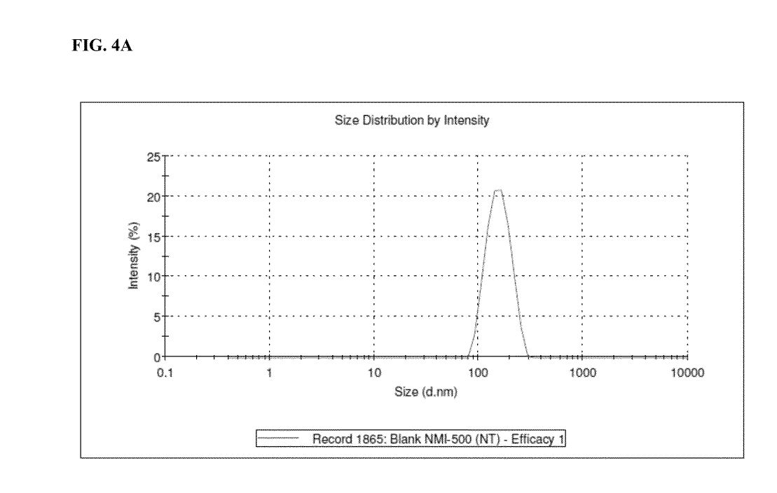

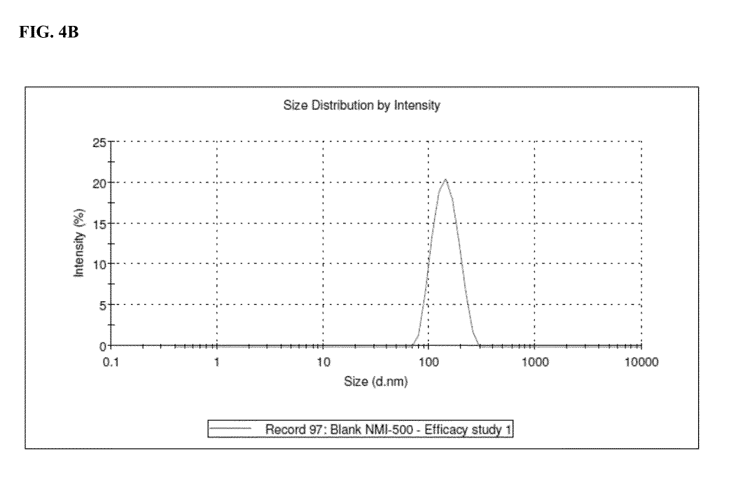

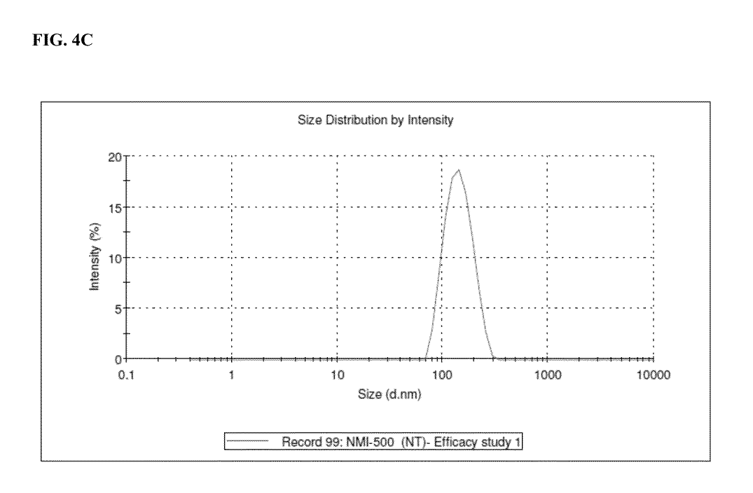

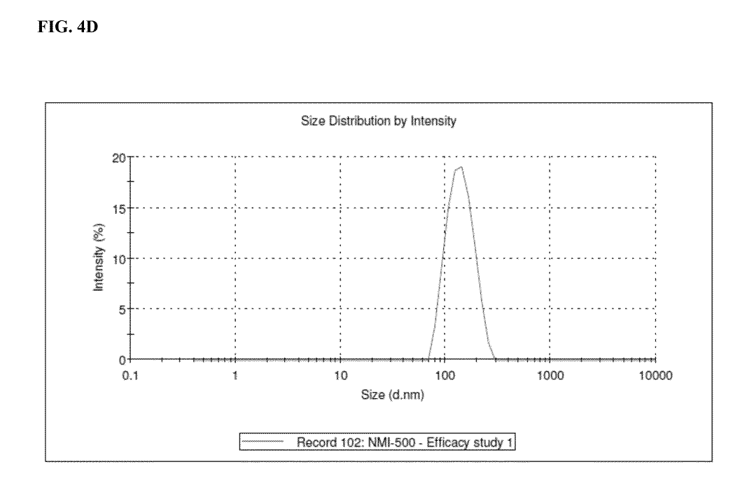

The particle size of these nanoemulsion formulations was determined using transmission electron microscopy (TEM) (FIGS. 3A-3D) and dynamic light scattering (DLS) plots (FIGS. 4A-4D) obtained from (Zetasizer ZS, Malvern Instruments Ltd., Worcestershire, United Kingdom). The particle size of these nanoemulsion formulations was below 150 nm in diameter.

The morphology of the oil droplets in the nanoemulsion formulations was visualized with transmission electron microscopy (TEM) analysis. TEM analysis was used to visualize any precipitation of the drug upon addition of the aqueous phase. Control, non-targeted, and folate receptor-targeted-containing nanoemulsion formulations (50 μL) were added to 200-mesh formware-coated copper TEM sample holders (EM Sciences, Hatfield, Pa., USA). The samples were then negatively-stained with 50 μL of 1% (w/v) uranyl acetate for 10 minutes at room temperature. Excess liquid was blotted with a piece of Whatman filter paper. The TEM samples were observed with JEOL 100-X transmission electron microscope (Peabody, Mass., USA) equipped with 20 μm aperture at 67 kV.

The TEMS shown in FIGS. 3A-3D indicate that the Gd-labeled, non-targeted nanoemulsion formulation not containing docetaxel, the Gd-labeled, folate receptor-targeted nanoemulsion formulation, the Gd-labeled, non-targeted nanoemulsion formulation containing docetaxel, and the Gd-labeled, folate receptor-targeted nanoemulsion formulation were all below 150 nm in diameter.

Nanoemulsion formulation size distribution and zeta potential values of control blank nanoemulsion formulation, non-targeted, and folate receptor-targeted nanoemulsion formulations were determined using Zetasizer ZS (Malvern Instruments, UK). The results are shown in below Table II. The average particle size of the control nanoemulsion formulation containing no docetaxel was below 150 nm in diameter. The incorporation of docetaxel in nanoemulsion formulations did not significantly change the hydrodynamic particle size and size remained below 150 nm. The average surface charge of the nanoemulsion formulations were in the range of −50 to −54 mV.

Additionally, the efficiency of docetaxel encapsulation in the nanoemulsion formulation was determined using an ultrafiltration method and docetaxel concentration was estimated using HPLC method, Table II. The results indicate that the total amount of docetaxel added to the preparation for encapsulation was in the oil phase of the nanoemulsion formulation.

TABLE II
Characterization of Nanoemulsion Formulation

| | Hydrodynamic | | | |
|---|---|---|---|---|
| | diameter | | Zeta | |
| | of formulations | | potential | Docetaxel |
| Test Samples | Size (nm) | PDI | (mV) | encapsulation |
| Control nanoemulsion | 142 ± 7 | 0.1 | −50 ± 10 | — |
| formulation no | | | | |
| docetaxel or folate | | | | |
| receptor-targeting agent) | | | | |
| Non-targeted | 130 ± 3 | 0.1 | −50 ± 12 | 100% |
| nanoemulsion | | | | |
| formulation containing | | | | |
| docetaxel | | | | |
| Folate receptor-targeted | 135 ± 6 | 0.06 | −54 ± 10 | 100% |
| nanoemulsion | | | | |
| formulation containing | | | | |
| docetaxel | | | | |

The DLS's shown in FIGS. 4A-4D indicate that the Gd-labeled, non-targeted nanoemulsion formulation not containing docetaxel, the Gd-labeled, folate receptor-targeted nanoemulsion formulation not containing docetaxel, Gd-labeled, non-targeted nanoemulsion formulation, and the Gd-labeled, folate receptor-targeted nanoemulsion formulation were all below 150 nm in diameter.

The nanoemulsion formulations of the present disclosure may have an interior and a surface, where the surface has a composition different from the interior, i.e., there may be at least one compound present in the interior but not present on the surface (or vice versa), and/or at least one compound is present in the interior and on the surface at differing concentrations. For example, a compound, such as a targeting moiety (i.e., a low-molecular weight ligand, protein, carbohydrate, or nucleic acid) of a polymeric conjugate of the present disclosure, may be present in both the interior and the surface of the nanoemulsion formulation, but at a higher concentration on the surface than in the interior of the nanoemulsion formulation, although in some cases, the concentration in the interior of the nanoemulsion formulation may be essentially nonzero, i.e., there is a detectable amount of the compound present in the interior of the nanoemulsion.

In some cases, the interior of the nanoemulsion formulation is more hydrophobic than the surface of the nanoemulsion formulation. For instance, the interior of the nanoemulsion formulation may be relatively hydrophobic with respect to the surface of the nanoemulsion formulation, and a drug or other payload may be hydrophobic, and readily associates with the relatively hydrophobic center of the nanoemulsion formulation. The drug or other payload can thus be contained within the interior of the nanoemulsion formulation, which can shelter it from the external environment surrounding the nanoemulsion formulation (or vice versa). For instance, a drug or other payload contained within a nanoemulsion formulation administered to a subject will be protected from a subject's body, and the body may also be substantially isolated from the drug for at least a period of time.

For example, an exemplary nanoemulsion formulation may have a PEG derivative corona with a density of about 1.065 g/cm3, or about 1.01 g/cm3 to about 1.10 g/cm3.

The nanoemulsion formulations of the present disclosure may have controlled release properties, e.g., may be capable of delivering an amount of active agent to a patient, for example to a specific site in a patient, over an extended period of time, for example over 1 day, 1 week, or more. Some disclosed nanoemulsion formulations substantially immediately release (for example over about 1 minute to about 30 minutes), less than about 2% in 6 hours, less than about 4% in 24 hours, less than about 7% in 48 hours, or less than about 10% of a chemotherapeutic agent (for example docetaxel) in 72 hours, for example when placed in a phosphate buffer saline solution at room temperature and/or at 37° C.

### 4. Methods of Treatment

The nanoemulsion formulations in accordance with the present disclosure may be used to treat, alleviate, ameliorate, relieve, delay onset of, inhibit progression of, reduce severity of, and/or reduce incidence of one or more symptoms or features of a cancer or tumor.

The term “cancer” includes pre-malignant as well as malignant cancers. Cancers include, but are not limited to, prostate, gastric cancer, colorectal cancer, skin cancer, e.g., melanomas or basal cell carcinomas, lung cancer, cancers of the head and neck, bronchus cancer, pancreatic cancer, urinary bladder cancer, brain or central nervous system cancer, peripheral nervous system cancer, esophageal cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. “Cancer cells” can be in the form of a tumor or exist alone within a subject (e.g., leukemia cells).

In certain cases, a targeted nanoemulsion may be used to treat any cancer where a folate receptor is expressed on the surface of cancer cells or in the tumor neovasculature, including the neovasculature of ovarian or non-ovarian solid tumors. Examples of the folate-related indication include, but are not limited to, breast, ovarian, esophageal, and oropharyngeal cancers.

When treating cancer, “therapeutically effective amount” of the nanoemulsion formulation of the present disclosure is administered to a patient and is that amount effective for treating, alleviating, ameliorating, relieving, delaying onset of, inhibiting progression of, reducing severity of, and/or reducing incidence of one or more symptoms or features of cancer.

As will be appreciated by those of ordinary skill in this art, the effective amount of the nanoemulsion formulation may vary depending on such factors as the desired biological endpoint, the drug to be delivered, the target tissue, the route of administration, etc. For example, the effective amount of the nanoemulsion formulation is the amount that results in a reduction in tumor size by a desired amount over a desired period of time. Additional factors which may be taken into account include the severity of the disease state; age, weight and gender of the patient being treated; diet, time and frequency of administration; drug combinations; reaction sensitivities; and tolerance/response to therapy.

The nanoemulsion formulations of the present disclosure can be used to inhibit the growth of cancer cells, for example, ovarian cancer cells. As used herein, the term “inhibits growth of cancer cells” or “inhibiting growth of cancer cells” refers to any slowing of the rate of cancer cell proliferation and/or migration, arrest of cancer cell proliferation and/or migration, or killing of cancer cells, such that the rate of cancer cell growth is reduced in comparison with the observed or predicted rate of growth of an untreated control cancer cell. The term “inhibits growth” can also refer to a reduction in size or disappearance of a cancer cell or tumor, as well as to a reduction in its metastatic potential. Such an inhibition at the cellular level may reduce the size, deter the growth, reduce the aggressiveness, or prevent or inhibit metastasis of a cancer in a patient. Those skilled in the art can readily determine, by any of a variety of suitable indicia, whether cancer cell growth is inhibited.

Inhibition of cancer cell growth may be evidenced, for example, by arrest of cancer cells in a particular phase of the cell cycle, for example, arrest at the G2/M phase of the cell cycle Inhibition of cancer cell growth can also be evidenced by direct or indirect measurement of cancer cell or tumor size. In human cancer patients, such measurements generally are made using well-known imaging methods, such as magnetic resonance imaging, computerized axial tomography and X-rays. Cancer cell growth can also be determined indirectly, such as by determining the levels of circulating carcinoembryonic antigen, prostate specific antigen or other cancer-specific antigens that are correlated with cancer cell growth inhibition of cancer growth is also generally correlated with prolonged survival and/or increased health and well-being of the subject.

Also provided herein are therapeutic protocols that include administering a therapeutically effective amount of a disclosed therapeutic nanoemulsion formulation to a healthy individual (i.e., a subject who does not display any symptoms of cancer and/or who has not been diagnosed with cancer). For example, healthy individuals may be “immunized” with an inventive targeted particle prior to development of cancer and/or onset of symptoms of cancer; at risk individuals (for example, patients who have a family history of cancer; patients carrying one or more genetic mutations associated with development of cancer; patients having a genetic polymorphism associated with development of cancer; patients infected by a virus associated with development of cancer; patients with habits and/or lifestyles associated with development of cancer; etc.) can be treated substantially contemporaneously with (for example, within 48 hours, within 24 hours, or within 12 hours of) the onset of symptoms of cancer. Of course individuals known to have cancer may receive inventive treatment at any time.

Nanoemulsion formulations disclosed herein may be combined with pharmaceutical acceptable carriers to form a pharmaceutical composition. As would be appreciated by one of skill in this art, the carriers may be chosen based on the route of administration as described below, the location of the target issue, the drug being delivered, the time course of delivery of the drug, etc.

For example, the nanoemulsion formulations of this disclosure can be administered to a patient by any means known in the art including oral and parenteral routes. The term “patient,” as used herein, refers to humans as well as non-humans, including, for example, mammals, birds, reptiles, amphibians, and fish. Sometimes parenteral routes are desirable since they avoid contact with the digestive enzymes that are found in the alimentary canal. These compositions may be administered by injection (for example, intravenous, subcutaneous, intramuscular, or intraperitoneal injection), rectally, vaginally, topically (as by powders, creams, ointments, or drops), or by inhalation (as by sprays).

For example, the nanoemulsion formulations of the present disclosure may be administered to a subject in need thereof systemically, for example, by intravenous infusion or injection.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parentally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono or di-glycerides. In addition, fatty acids such as oleic acid are used in the preparation of injectables.

It will be appreciated that the exact dosage of the nanoemulsion of the present disclosure is chosen by the individual physician in view of the patient to be treated, in general, dosage and administration are adjusted to provide an effective amount of the nanoemulsion formulation to the patient being treated.

The nanoemulsion formulations of the present disclosure may be formulated in dosage unit form for ease of administration and uniformity of dosage. The expression “dosage unit form” as used herein refers to a physically discrete unit of the pharmaceutical formulation appropriate for the patient to be treated. It will be understood, however, that the total daily usage of the nanoemulsion formulation of the present disclosure will be decided by the attending physician within the scope of sound medical judgment. For any nanoemulsion formulation, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of the nanoemulsion formulation can be determined by standard pharmaceutical procedures in cell cultures or experimental animals, for example, ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions, which exhibit large therapeutic indices, may be useful in some embodiments. The data obtained from cell culture assays and animal studies can be used in formulating a range of dosage for human use.

For example, the nanoemulsion formulation may contain a docetaxel compound at a concentration of about 0.001% to 0.5% (0.01 mg/ml to 5 mg/ml). The dosage administered by injection may contain docetaxel in the range of about 75 mg to 1000 mg given as a one-hour infusion on the first day of each cycle (every 3 weeks) over 10 cycles depending upon the patient. A useful dosage can be about 100 mg in the first day of each cycle up to 10 cycles to a patient having a body weight of about 40 kg to 100 kg. Such dosages may prove useful for patients having a body weight outside this range.

Nanoemulsion formulations for oral administration may be of about the same volume as those used for injection. However, when administering the drug orally, higher doses may be used when administering by injection. For example, a dosage containing about 100 mg to 150 mg docetaxel in the first day of every 3 weeks may be used. In preparing such a dosage form, standard making techniques may be employed.

### 5. Methods of Imaging

The nanoemulsion formulations in accordance with the present disclosure may be used to image tumors or cancer cells. These nanoemulsion formulations are small enough to travel into minute body regions and, when coupled with paramagnetic elements, such as gadolinium ions (Gd³⁺), iron oxide, iron, platinum, or manganese, can enhance tissue contrast in MRI. Once the nanoemulsion formulation has reached the cancer site, its efficacy is determined, which can be done using an in vivo imaging modality such as MRI. Image-guided therapy using nanoemulsion formulations couples drug delivery with tissue imaging to allow clinicians to efficiently deliver chemotherapeutic agents, while simultaneously localizing the drugs and visualizing their physiological effects.

The nanoemulsion formulations combined with an appropriate imaging agent can act as MRI contrast agents to enhance tissue image resolution. Contrast agents such as Gd³⁺ have unpaired electrons that interact with surrounding water molecules to decrease their proton spin time, also referred to as T₁. Relaxation time is defined as the period it takes for a proton to return to its equilibrium position following a magnetization pulse. MRI can measure T₁ by creating a magnetic field that reverses the sample's magnetization, and then recording the time required for the spin directions to realign in their equilibrium positions again. The decreased T₁ relaxation time of the target tissue allows an MRI machine to better distinguish between it and its surrounding aqueous environment.

The nanoemulsion formulations according to the disclosure can serve as a new Gd³⁺ chelated, folate receptor-targeted nanoemulsion formulation that not only exhibits MRI contrast but also carries encapsulated docetaxel compounds to the target tissue for successful image-guided therapy. To examine the MRI contrast potential of these nanoemulsions, in vivo studies were conducted using MRI, while cell uptake and trafficking as well as efficacy studies were conducted to examine the drug delivery potential of the nanoemulsion formulation.

The method of imaging includes administering to a patient or subject to be imaged a diagnostically effective amount of a nanoemulsion formulation according to the disclosure. The nanoemulsion formulation can be administered by a variety of techniques including subcutaneously and intravenously. The method is effective for imaging cancers, such as breast, ovarian, esophageal, and oropharyngeal cancers and other cancers accessible by the lymphatic or vascular (blood) systems. For magnetic resonance imaging methods, the nanoemulsion formulation of the disclosure includes a paramagnetic metal ion (e.g., Gd³⁺).

The following examples provide specific exemplary methods of the invention, and are not to be construed as limiting the invention to their content.

### EXAMPLES

### Example 1

### Synthesis of Folate-Targeting Ligand DSPE-PEG-Cys-FA

The 1,2-distearoyl-sn-glycero-3-phosphoethanolamine-polyethylene-glycol-cysteine-folate (DSPE-PEG-Cys-FA) complex was prepared according to the scheme in FIG. 5. Briefly, 1,2-distearoyl-sn-glycero-3-phosphoethanolamine-polyethylene-glycol-malemide (DSPE-PEG-Mal) (100 mg, 1.3596 mM) was added to cysteine (8.24 mg, 2.72 mM) in a 1:2 molar ratio in HEPES buffer (25 mL) and the coupling reaction to create DSPE-PEG-Cys was carried out overnight at 4° C. under a nitrogen environment. The next day, excess cysteine was dialyzed out for 24 hr using 2 kD cut-off dialysis bags. The outside water was changed every 2 hr to facilitate dialysis.

A purified sample was freeze-dried and characterized by NMR. In the next step 51 mg of DSPE-PEG-Cys was dissolved in 6 mL dry DMSO containing 13 mg folic acid. 3 ml pyridine was added to the solution followed by 16 mg of N,N′-dicyclohexylcarbodiimide. The coupling was carried out for 4 hr at room temperature (RT) with continuous mixing. The sample was dialyzed in water using 2 kD cut-off dialysis bags. Outside water was changed every 2 hr for 24 hr to facilitate dialysis. Purified sample was freeze-dried and characterized by NMR.

### Example 2

### Synthesis of Gd⁺³-DTPA-PE

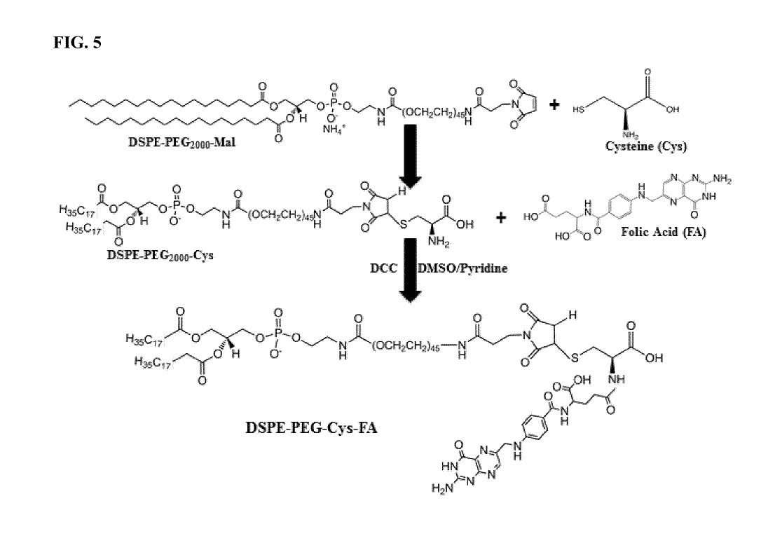

The gadolinium diethylene triaminepentacetic acid-phosphatidyl ethanolamine complex (Gd⁺³-DTPA-PE) chelate was prepared according to the scheme shown in FIG. 6. Briefly, 30 μl of triethylamine (Sigma) was added to 100 mg L-α-phosphatidylethanolamine, transphosphatidylated (egg chicken) (841118C, Avanti Polar Lipids, Birmingham, Ala.) dissolved in 4 ml chloroform (extra dried). This solution was then added drop-wise to 400 mg (1 mM) diethylene triaminepentacetic dianhydride (DTPA anhydride) (Sigma-Aldrich, Natick, Mass.) in 20 ml dimethyl-sulfoxide, and the mixture was stirred for 3 hr under nitrogen atmosphere at RT. Nitrogen was then blown on to a sample to remove the chloroform.

The DTPA-PE conjugate was then purified by dialysis against deionized distilled water at RT using a 3 kD molecular weight cut-off membrane (Spectrapore, Spectrum Laboratories, Rancho Dominguez, Calif.). The purified sample was then transferred into tubes and freeze-dried for 48 hr. The DTPA-PE complex formation and purity of the complex were monitored by thin layer chromatography (TLC) using a mobile phase of chloroform:methanol:water at a 3.25:1.25:0.5 (v/v) ratio and using ninhydrin as a visualizing reagent. For this, reactants (DTPA, PE) and complex (DTPA-PE) were dissolved in chloroform, placed on a TLC plate, and developed in the mobile phase. Ninhydrin solution was then sprayed, and the spots and their retention times were compared for the formation of the complex.

18.5 mg (10.0 mM) gadolinium (III) chloride hexahydrate (Sigma) in 0.1 ml water was then added drop-wise to the 100 mg DTPA-PE complex dissolved in 20 ml DMSO, and the reaction mixture was stirred (400 rpm) for 1 hr.

An Arsenazo III assay (see e.g., Serhan et al. (1981) J. Biol. Chem. 256:2736-2741) was used to monitor the reaction and formation of the Ge-DTPA-PE complex. 10 μl reaction mixture was added to 0.2 mM Arsenazo III (Pointe Scientific) in water and observed for the color change (pink to blue). No change in solution color indicated that all Ge was chelated by the DTPA-PE (free Gd³⁺ turns Arsenazo III solution to a blue color).

The resulting Gd³⁺-DTPA-PE conjugate was purified by dialysis against deionized distilled water at RT using a 3 kD molecular weight cut-off membrane (Spectrapore, Spectrum Laboratories). The purified sample was then transferred into tubes and freeze-dried for 48 hr. The Gd³⁺-DTPA-PE conjugate (˜100 mg) was stored at −20° C. until use.

### Example 3

### Preparation of Folate Receptor-Targeted, Gd, Docetaxel Nanoemulsion Formulations

The folate receptor-targeted, Gd³⁺, docetaxel nanoemulsion formulation was prepared accordingly to the method shown in FIG. 2.

Briefly, the oil phase was prepared as follows: 10 mg docetaxel was dissolved in 2 ml chloroform (extra dry) and added to 1 g flax seed oil in a glass scintillation vial. Nitrogen gas was blown for 30 min on the sample to remove chloroform and to form the oil phase with docetaxel.

The aqueous phase of this therapeutic formulation was prepared as follows: 100 mg Gd-DTPA-PE, 8 mg DSPE-PEG-Cys-Folate complex, 120 mg egg lecithin (Lipoid E 80, Lipoid GMBH, Ludwigshafen, Germany), and 15 mg PEG₂₀₀₀DSPE (Lysan Bio Inc., Arab, Ala.) were added to 4 ml 2.21% w/v glycerol (Sigma) in a glass scintillation vial made in water for injection. The mixture was stirred for 0.5 hr at 400 rpm to achieve complete dissolution of these conjugates.

The aqueous and oil phases from above steps were heated to 60° C. for 2 min in a water bath, and the aqueous phase was then added and vortexed for 1 min. The resulting mixture was passed through a LV1 Microfluidizer (Microfluidics Corp., Newton, Mass.) at 25,000 psi for 10 cycles.

These steps resulted in the production of a stable folate receptor-targeted, Gd-labeled-docetaxel nanoemulsion formulation. Each batch produced 5 ml of nanoemulsion formulation.

### Example 4

### Stability Studies of Nanoemulsion Formulations in Plasma and Intravenous Infusion Solutions

The physical stability of nanoemulsion formulation can be affected upon iv injection or mixing in iv infusion solutions ranging from particle aggregation to disruption. Aggregation leads to rapid in vivo clearance, whereas particle disruption causes burst release kinetics of payload (dose dumping). Physical stability of nanoemulsion formulation in plasma and parenteral infusion solutions was evaluated by measuring its particle size distribution over 24 hr.

Nanoemulsion formulations were diluted in plasma and intravenous (iv) infusion solutions and monitored for particle size over 24 hr at 37° C.

The change in particle size was analyzed by DLS and used as indicator of stability upon dilution. For this example, nanoemulsion formulations were diluted with fresh dog plasma, NaCl (0.9%), dextrose (5%), or phosphate buffered saline (PBS, pH 7.4) to form mixtures containing 10% nanoemulsion formulation. The mixtures were then incubated at 37° C. 10 μl of samples were taken for analysis at 1 hr, 2 hr, 4 hr, 6 hr, 8 hr, and 24 hr, diluted 1000-fold with distilled water, and the particle size was analyzed using a Zetasizer ZS as described above.

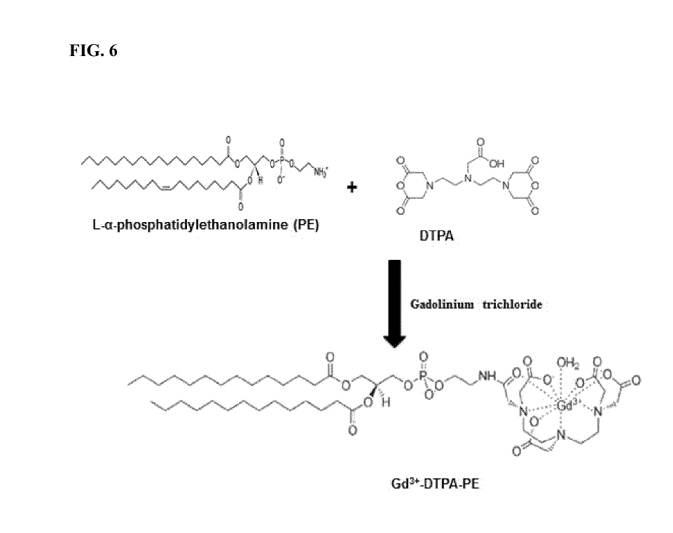

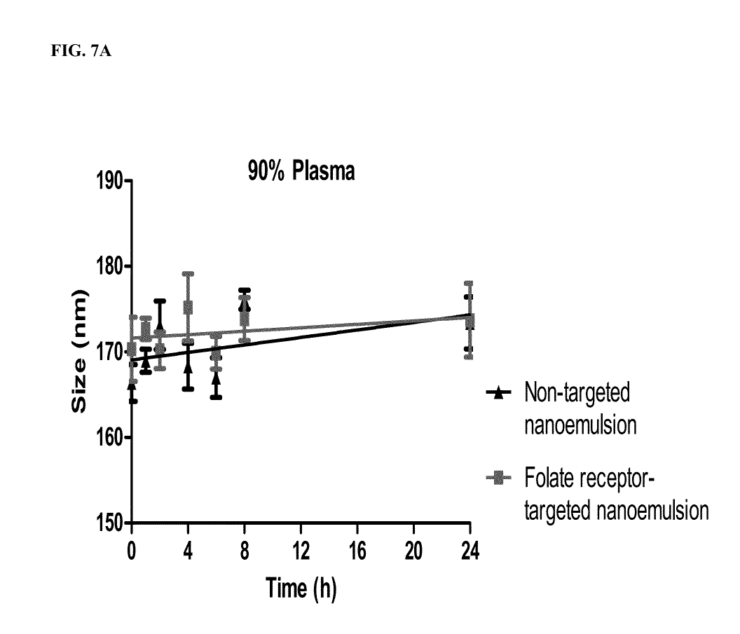

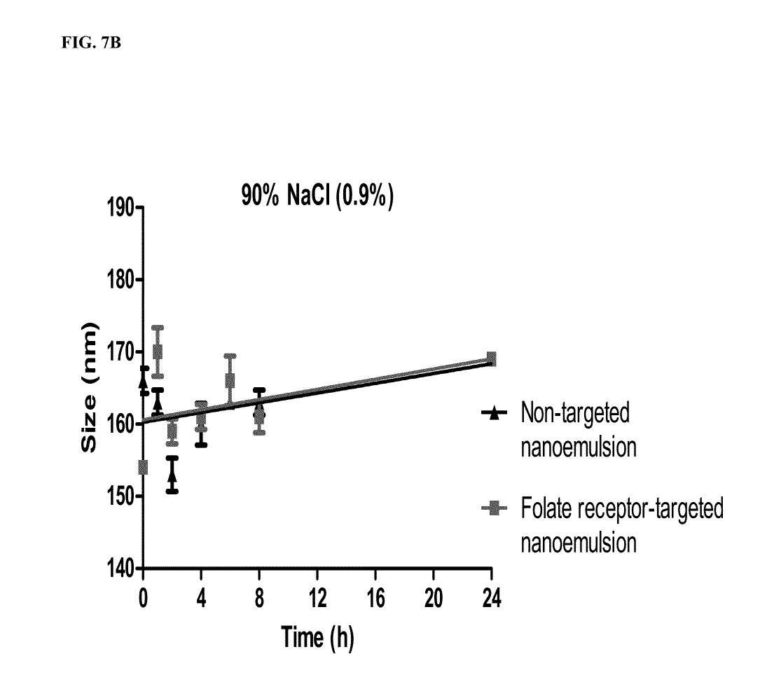

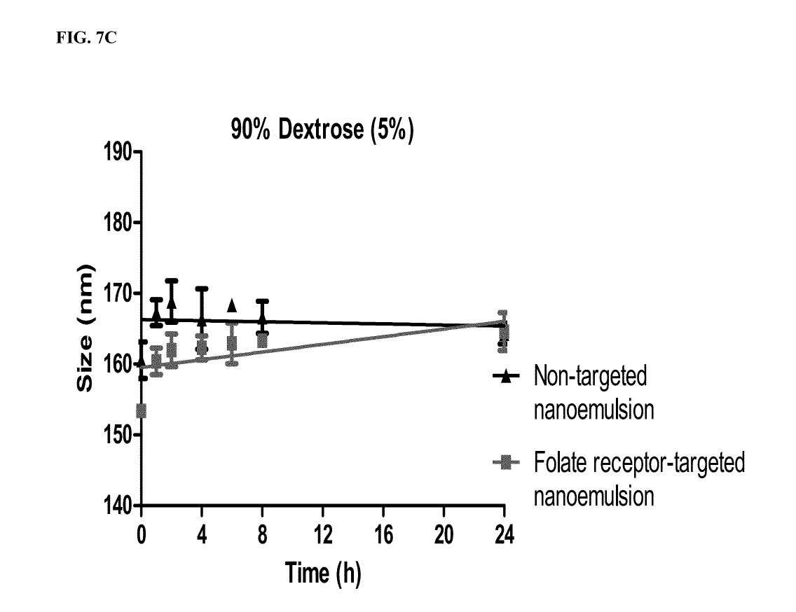

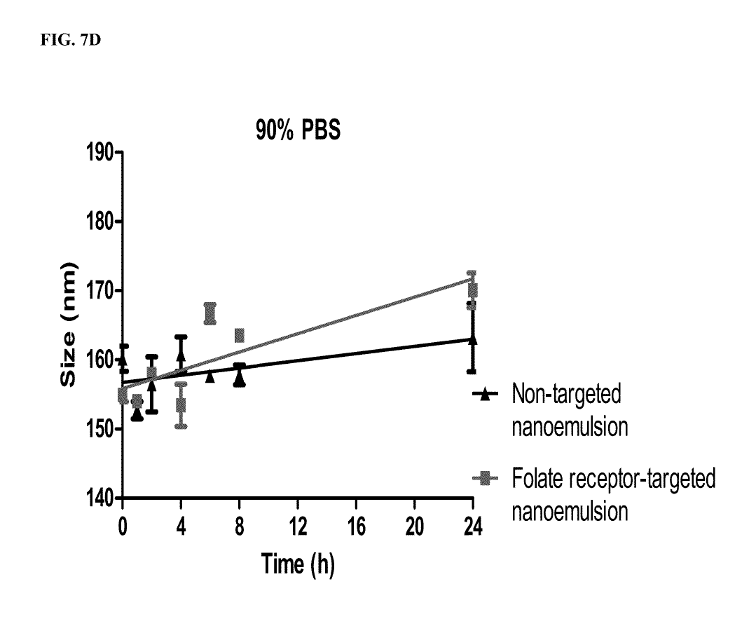

The results shown in FIGS. 7A-7D indicate that the particle size did not change significantly (P>0.05) over a 24 hr period, indicating that both non-targeted and folate receptor-targeted nanoemulsion formulations were intact, and that there was no particle aggregation and disruption in presence of high electrolyte concentration and plasma proteins.

These data suggest that the nanoemulsion formulations are stable in vivo in blood circulation, have longer residence time in the blood stream, and have enhanced ability to accumulate in tumors through the enhanced permeability and retention effect.

### Example 5

### Drug Release Studies of Nanoemulsion Formulations

Assays, which measure docetaxel release from nanoemulsion formulations, were carried out as follows. Nanoemulsion formulations consisting of 4 mg of docetaxel were loaded into dialysis bags with a molecular weight cut-off 3.5 kD (Spectrapore, Spectrum Laboratories, Rancho Dominguez, Calif.) and placed in 100 ml of release media (0.5% Tween 80/Phosphate buffered saline, pH 7.4) maintained at 37° C. and stirred at 400 rpm. Sample aliquots (1 ml) were collected at different time intervals up to 72 hr and replaced with fresh media. The docetaxel concentration in the samples was analyzed using HPLC. At the end of study, the unreleased docetaxel remaining in the dialysis bag was measured and compared with the release data. The cumulative amount of docetaxel released and release kinetics were calculated based on the observed release profile.

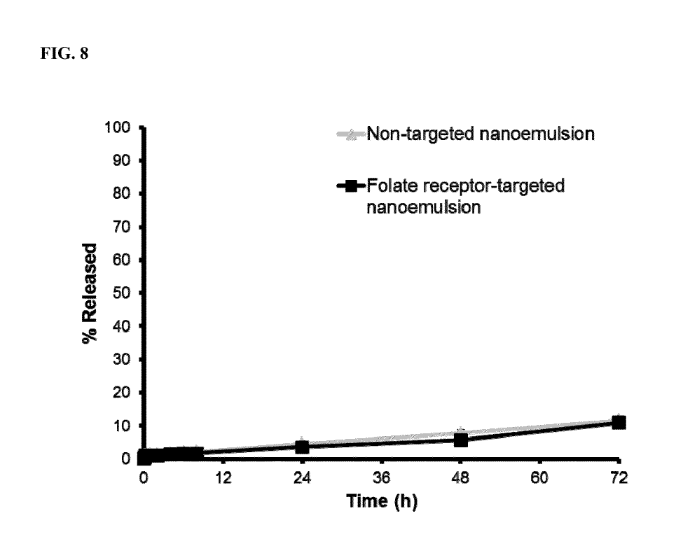

The results of the release assay are shown in FIG. 8. Predominant docetaxel release mechanism for non-targeted and folate receptor-targeted nanoemulsion formulations followed zero order kinetics (Lachman, et al. (1986) “The Theory and Practice of Industrial Pharmacy”. Lea & Febiger.” Philadelphia. USA).

### Example 6

### Cellular Uptake Studies

A useful assay, which demonstrates the effect that targeted nanoemulsion formulations have on cellular uptake, is as follows. The uptake was measured in ovarian SKOV3 cells using fluorescence. SKOV3 cells growing on cover slips in 6-well plate at 3000 cells/well were incubated with the fluorescently labeled nanoemulsion formulations for 5 min, 15 min, 30 min and 60 min. At the end of incubation period, cells were washed thrice with phosphate buffered saline (PBS) and incubated with Lyso Tracker and DAPI for 10 min, which stains lysosomes and nucleus of the cells, respectively. Cells were further washed with PBS, inverted and mounted on glass slides using Flouromount G mounting media.

0.01% NBD-Ceramide (green) at 0.01% w/v was incorporated into all nanoemulsion formulations as a fluorescent dye. 0.02% folate was incorporated into the targeted nanoemulsion formulations. Lyso Tracker (red) and DAPI (blue) were used to monitor the co-localization of the nanoemulsion formulations in the SKOV3 cells. Images were acquired using a Confocal Zeiss LSM 700 microscope with an object 63× oil immersion over a 60 min period.

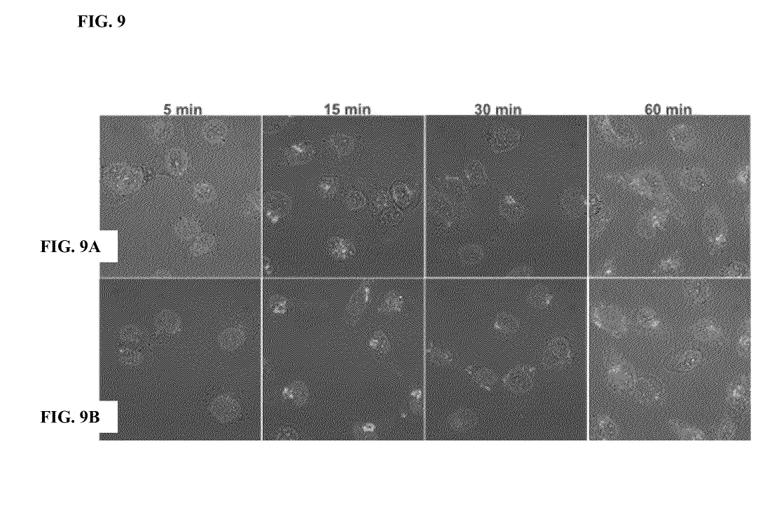

The images in FIG. 9A show that NBD-CER (green) found in non-targeted nanoemulsion formulations co-localized with Lyso Tracker (red), indicating the entry of NBD-CER and therefore the non-targeted nanoemulsion formulation into lysosomes.

In contrast, the images in FIG. 9B show that NBD-CER (green) found in folate receptor-targeted nanoemulsion formulations does not co-localize with Lyso Tracker (red), indicating the entry of NBD-CER and therefore the folate receptor-targeted nanoemulsion formulation into endosomes.

This study demonstrates that the folate receptor-targeted nanoemulsion formulations of the present disclosure are able to evade the drug degradative lysosomal pathway, enhancing drug concentrations in cells in vitro.

### Example 7

### In Vitro Caspase 3/7 Assay

The Caspase 3/7 activity is a pathway that is stimulated during apoptosis. That the nanoemulsion formulations of the present disclosure are useful in stimulating apoptosis was demonstrated using a Caspase 3/7 assay.

Skov-3 or Skov-3_{TR} cells were seeded in 96-well microplates at a density of 20,000 cells/well. They were then treated with (1) a non-targeted nanoemulsion formulation, (2) a folate receptor-targeted nanoemulsion, (3) Taxotere®, or (4) vehicle controls with Taxotere® ((1 ml) prepared to contain 20 mg docetaxel in 50/50 (v/v) ratio of polysorbate 80 to dehydrated alcohol). The cells were washed thrice with PBS buffer to remove any formulation that did not enter the cells, and were then treated with Apo-ONE Caspase-3/7 substrate (Promega, Madison, Wis.) solution (100 gl/well). The fluorescence of each well was measured at excitation wavelength of 490 nm and emission wavelength of 520 nm using a Synergy HT microplate reader (Biotek Instruments, Winooski, Vt.).

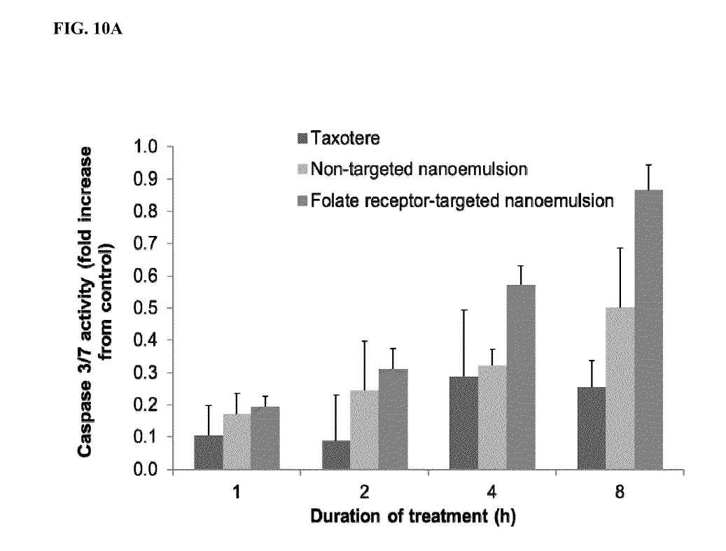

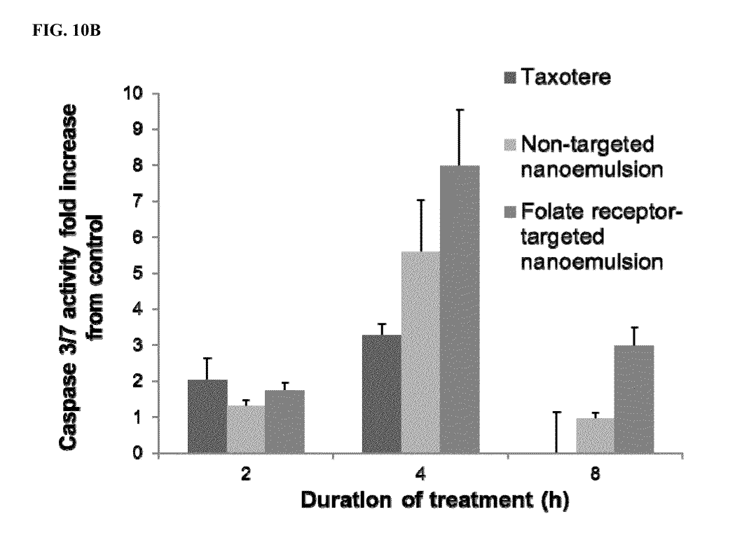

FIG. 10A displays the increase in caspase 3/7 activity in SKOV-3 cells following the treatment with the non-targeted nanoemulsion formulation, the folate receptor-targeted nanoemulsion, or with Taxotere®, relative to control (untreated cells), measured at 1 hr, 2 hr, 4 hr, and 8 hr. Increased apoptotic activity was observed when the docetaxel was encapsulated in the non-targeted nanoemulsion compared to the Taxotere. However, a higher apoptotic activity was observed with the targeted nanoemulsion formulations of this disclosure that comprised a folate receptor-targeting ligand.

FIG. 10B shows the caspase 3/7 activity in SKOV-3_{TR} cells following the treatment with non-targeted nanoemulsion formulation, folate receptor-targeted nanoemulsion formulation and Taxotere®, relative to control (untreated cells), measured after the 2 hr, 4 hr, and 8 hr. Increased apoptotic activity was observed at 4 hr and 8 hr of treatment with non-targeted nanoemulsion formulation and folate targeted nanoemulsion formulation compared to the Taxotere. SKOV-3TR cells highly express p-glycoprotein (Pgp), a multi-drug resistant transporter. The higher apoptotic activity seen with the folate receptor-targeted nanoemulsion formulation indicates that the nanoemulsion formulations with folate receptor-targeting ligands bypass the drug efflux Pgp pump and enhance targeted nanoemulsion uptake inside the cells leading to higher efficacy.

Thus, nanoemulsion formulations of the disclosure are useful to stimulate apoptosis of cancer cells.

### Example 8

### Determination of Maximum Tolerated Dose of Nanoemulsion Formulations

In order to determine the maximum tolerated dose (MTD) of nanoemulsion formulations according to the disclosure was done. Female Nu/Nu mice (3 mice/group) were treated with 7.5 mg/kg, 10 mg/kg, and 16 mg/kg docetaxel in a non-targeted nanoemulsion formulation, a folate receptor-targeted nanoemulsion formulation, or with Taxotere. Non-targeted nanoemulsion formulation, folate receptor-targeted nanoemulsion formulation, or Taxotere was intravenously via tail vein using BD tuberculin syringe attached with 27 gauge needle on day 0 and day 4. The body weights of the treated mice were measured daily until day 8 and compared to the control (untreated) group. Mice in all groups were treated with pemetrexed intravenously (400 μg/mouse in 100 μl of 0.9% sodium chloride solution) 1 hr prior to treatment. Pemetrexed blocks folate receptors in kidney, thereby preventing folate receptor-targeted nanoemulsion formulation accumulation in the kidney. The dose that causes mice to lose ≧10% of their initial body weight is considered the MTD.

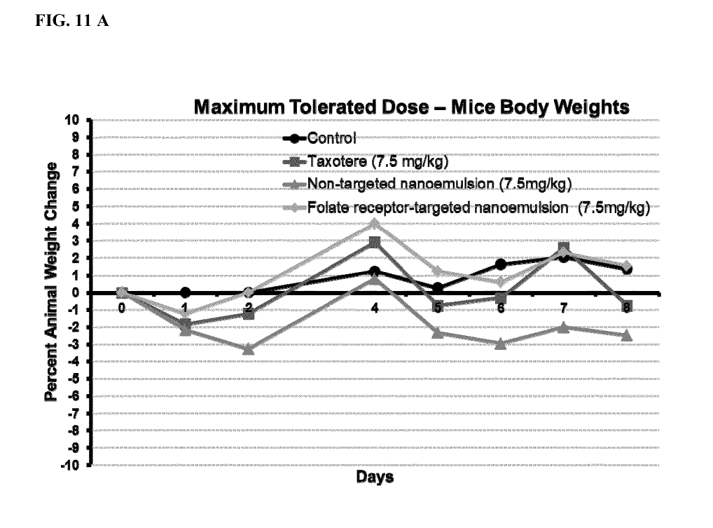

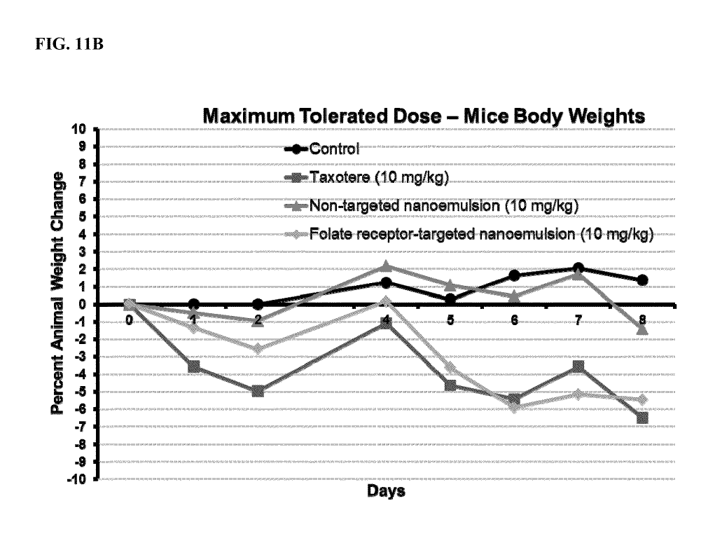

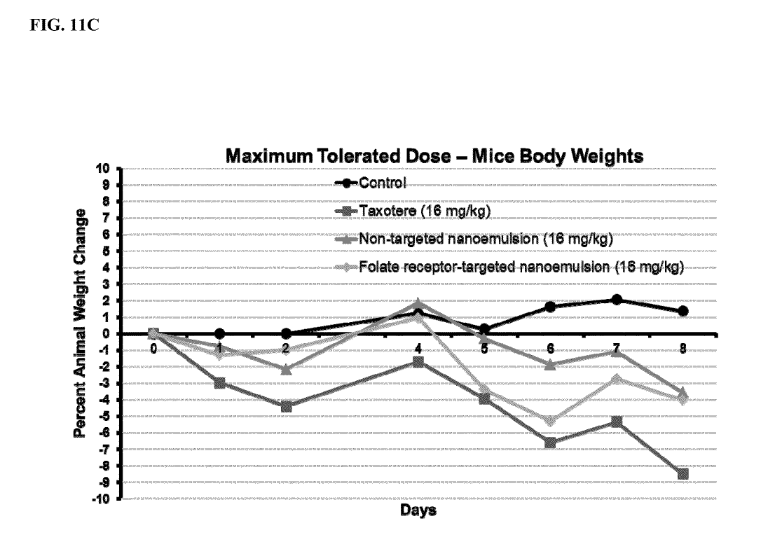

The results shown in FIGS. 11A-11B demonstrate that mice tolerated both 7.5 mg/kg and 10 mg/kg nanoemulsion formulation. However, at 16 mg/kg (FIG. 11C), the mice in the Taxotere group lost around 10% body weight, as compared to mice treated with non-targeted or folate receptor-targeted nanoemulsion formulations (4% and 5%), respectively. These data demonstrate that the mice tolerate higher doses of docetaxel in non-targeted and folate receptor-targeted nanoemulsion formulations of this disclosure than without such nanoemulsion formulation delivery.

### Example 9

### In Vivo Pharmacokinetic Studies of Nanoemulsion Formulations

A dose range finding (DRF) study was conducted to determine the pharmacokinetic profile of folate receptor-targeted nanoemulsion formulations according to the disclosure administered to CD-1 mice at dose levels ranging from 1 mg/kg to 10 mg/kg. CD-1 mice were injected intravenously with folate receptor-targeted nanoemulsion formulation. Blood was collected at 5 min, 30 min, 1 hr, 2 hr, 4 hr, 6 hr, 8 hr, 12 hr, and 24 hr after injection. Docetaxel concentration in the plasma was measured using a liquid chromatography-mass spectrometer (LC-MS/MS).

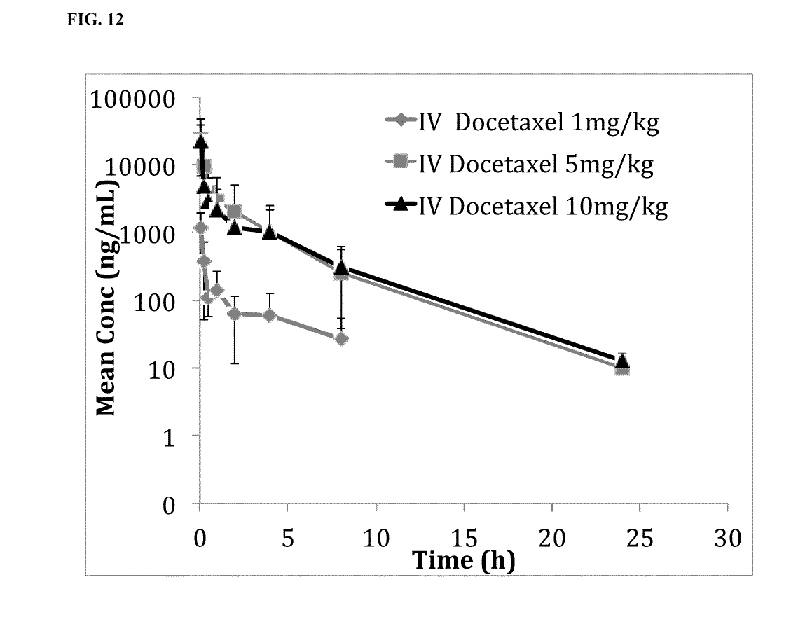

The results shown in FIG. 12 demonstrate that the blood concentration of folate receptor-targeted nanoemulsion formulation increases from 1 mg/kg to 5 mg/kg of docetaxel, but there is no further increase upon raising the concentration from 5 mg/kg to 10 mg/kg.

Pharmacokinetic parameters were calculated by non-compartmental analysis using Phoenix WinNonlin 6.2 version. The results are shown in Table III.

TABLE III
Plasma Pharmacokinetic Profile of Docetaxel from Dose Range Finding Study

| | Tmax | Cₘₐₓ | t_{1/2} | AUCₗₐₛₜ | AUC_{0-∞} | Vd | Cl | MRT |
|---|---|---|---|---|---|---|---|---|
| Docetaxel | (h) | (ng/ml) | (h) | (h*ng/ml) | (h*ng/ml) | (ml/kg) | (ml/h/kg) | (h) |
| 1 mg/kg | 0.083 | 2125 ± 1181 | 4.29 ± 2 | 789 ± 667 | 475 ± 34 | 13275 ± 7029 | 2110 ± 153 | 1.53 ± 0.3 |
| 5 mg/kg | 0.083 | 38267 ± 31757 | 4.9 ± 2.6 | 19026 ± 24077 | 19095 ± 24050 | 9074 ± 12083 | 922 ± 1006 | 2.25 ± 0.5 |
| 10 mg/kg | 0.083 | 52229 ± 28251 | 3.7 ± 1 | 16653 ± 28251 | 16719 ± 28251 | 5801 ± 5151 | 962 ± 752 | 2.6 ± 1 |

Docetaxel exhibited linear pharmacokinetics from 1 mg/kg to 5 mg/kg; whereas a somewhat similar profile was observed with 5 mg/kg and 10 mg/kg dosing.

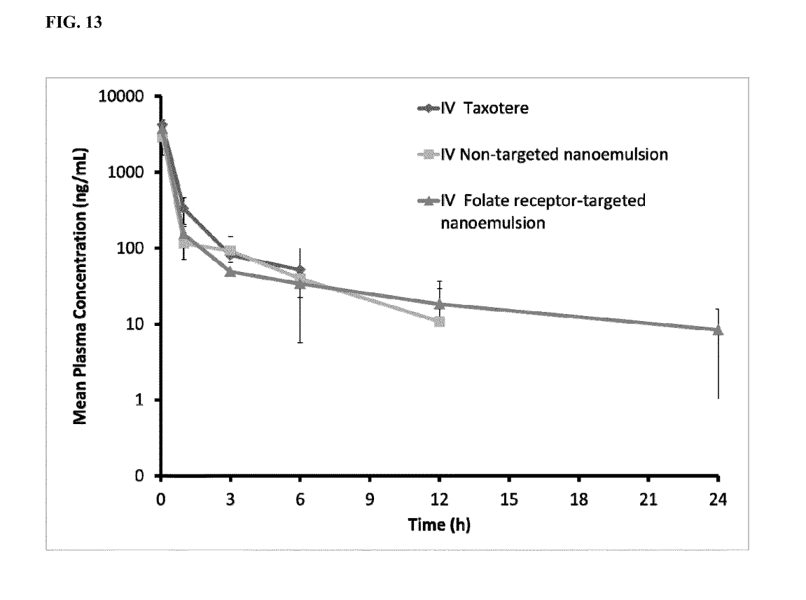

In another experiment, Nu/Nu mice were injected intravenously with docetaxel at 10 mg/kg as Taxotere, non-targeted nanoemulsion formulation, or folate receptor-targeted nanoemulsion formulations, and blood collected at 5 min, 30 min, 1 hr, 2 hr, 4 hr, 6 hr, 8 hr, 12 hr, and 24 hr after injection. Mice in all groups were treated with pemetrexed intravenously (400 μg/mouse in 100 μl of 0.9% sodium chloride solution) 1 hr prior to treatment. Docetaxel concentration in the plasma samples was determined by LC-MS, and a time vs. concentration time profile was prepared using Microsoft Excel™ software. The results are shown in FIG. 13, which displays the plasma pharmacokinetic profile of docetaxel in nude mice.

Docetaxel pharmacokinetic parameters were calculated by non-compartmental analysis using Phoenix-WinNonlin 6.2 version. The results are shown in Table IV.

TABLE IV
Plasma Pharmacokinetic Profile of Docetaxel

| | Tmax | Cmax | t½ | AUC0-∞ | Vd | Cl | MRT |
|---|---|---|---|---|---|---|---|
| Test Sample | (h) | (ng/L) | (h) | (h*ng/L) | (L/kg) | (L/h/kg) | (h) |
| Taxotere | 0.083 | 4207 ± 670 | 1.4 ± 0.3 | 3195 ± 412 | 6.4 ± 1.8 | 3.2 ± 0.4 | 0.9 ± 0.4 |
| Non-target- | 0.083 | 2930 ± 1266 | 3.2 ± 0.7 | 2328 ± 614 | 20.8 ± 6.6 | 4.5 ± 1.2 | 1.9 ± 1.0 |
| nanoemulsion | | | | | | | |
| formulation | | | | | | | |
| Folate-targeted | 0.083 | 3660 ± 433 | 6.1 ± 3.8 | 2840 ± 55 | 31 ± 19 | 3.5 ± 0.1 | 3 ± 2.1 |
| nanoemulsion | | | | | | | |
| formulation | | | | | | | |

These results show that the nanoemulsion formulation extended the biological half-life of docetaxel in the blood.

### Example 10

### Efficacy and Safety of Nanoemulsion Formulations

In order to determine if the nanoemulsion formulations according to the disclosure produce a cytotoxic effect, the following experiments were done on SKOV3 and SKOV3_{TR} cells. SKOV3_{TR} cells express P-glycoprotein (Pgp), which produces chemotherapeutic agent efflux out of the cell and is associated with multidrug resistant cancer cells.

A tetrazolium (MTT) assay was performed, which measures the activity of cellular enzymes that reduces the MTT dye to insoluble formazan. SKOV3 and SKOV3_{TR} cells were treated with corresponding test nanoemulsions or control solutions as indicated below in Table V. Polyethylenimine at 50 μg/ml was used as a positive control for cytotoxicity. The effect of docetaxel in solution, a nanoemulsion formulation, and a folate receptor-targeted nanoemulsion formulation of the disclosure on the viability of ovarian SKOV3 and SKOV3_{TR} cells was studied and measured after 72 hr treatment. After the completion of treatment, cells were incubated with MTT reagent (50 μg/well) for 2 hr, the resulting formazan crystals were dissolved in dimethyl sulfoxide (150 μg/well) and measured at 570 nm in the Plate reader (Synergy HT, Biotek Instruments, Winooski, Vt.).

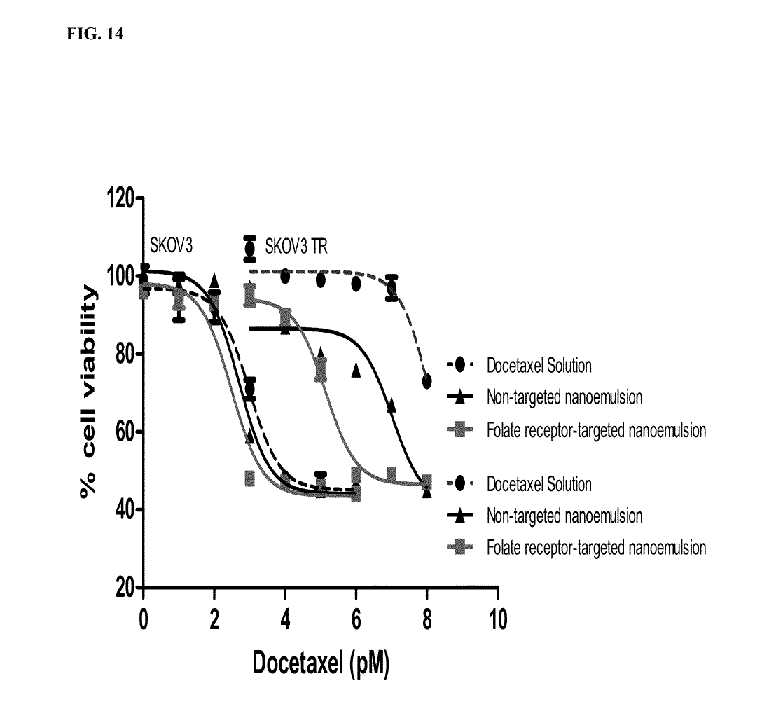

The concentration of drug that inhibits fifty percent of growth is known as the 50% growth inhibitory concentration (IC₅₀). Using the dose response data shown in FIG. 14, the IC₅₀ was calculated and are shown in Table V. Values are shown as mean±SD, n=8. All IC₅₀ values were obtained by analyzing the MTT assays results using Graphpad Prism 5 scientific data analysis software.

TABLE V
Inhibitory Concentration Analysis

IC₅₀ (nM)

| Treatment | SKOV3 | SKOV3TR |
|---|---|---|
| Docetaxel in solution | 1.0 ± 1.3 | 27,000 ± 4.5 |
| Docetaxel non-targeted | 0.5 ± 1.2 | 6,800 ± 1.4 |
| nanoemulsion formulation | | |
| Docetaxel folate receptor- | 0.3 ± 1.2 | 100 ± 1.2 |
| targeted nanoemulsion | | |
| formulation | | |

As shown in Table V, IC₅₀ was decreased when non-targeted nanoemulsion formulations of the present disclosure were used. However, a greater decrease in IC₅₀ was observed with the addition of folate receptor-targeting ligands to the nanoemulsion formulations. Most significant is the decrease observed when SKOV3_{TR} cells were treated with folate receptor-targeted nanoemulsion formulations of the present disclosure, indicating that the folate receptor-targeted nanoemulsion formulations are capable of by-passing the multidrug resistant mechanisms that these cells express. The nanoemulsions containing no docetaxel did not affect cell viability.

In order to determine if the nanoemulsion formulations according to the disclosure produce had therapeutic efficacy against cancer in vivo the following experiment was performed.

Orthotropic tumors were developed in 100 Nu/Nu female mice, each mouse weighing approximately 20 g (Charles River Labs, Cambridge, Mass.). The mice were injected subcutaneously with 1×10⁶ SKOV3 human ovary cancer cells (ATCC, Manassas, Va.) suspended in phosphate buffered saline. The 100 mice were divided into 5 test groups of 20 individual mice. Each mouse was then dosed 7 days a week for 6 weeks with either nothing (control group) or 1 of 2 test compounds. The first test compound was Taxotere (free docetaxel), which was administered as a comparative sample in 10 mg/kg doses. The second test compound was a folate receptor-targeted nanoemulsion formulation. Docetaxel was present in the test samples at 2 mg/ml and was administered as a comparative sample in 10 mg/kg doses. The survival time of each group of mice was determined and the median survival time (days) calculated using Graphpad Kaplan-Meyers survival analysis software on the basis of the observed survival time of each mouse.

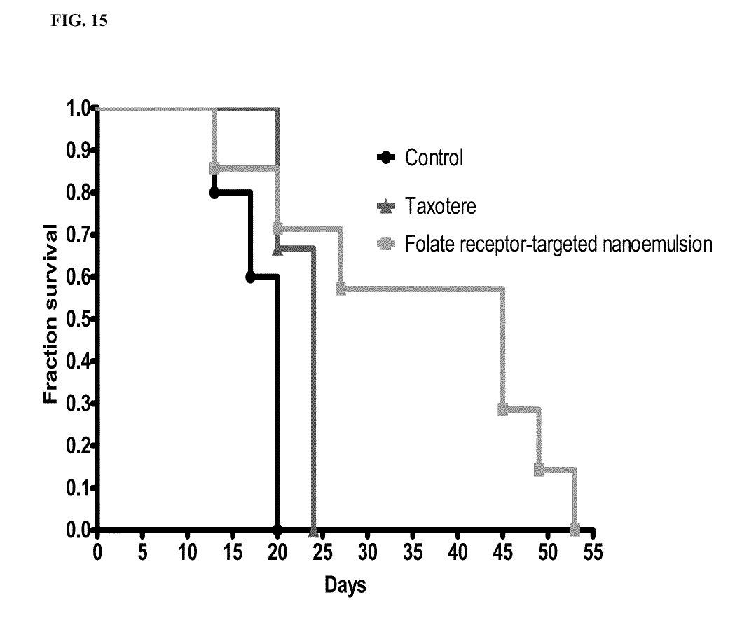

The results in FIG. 15 show that the fractional survival for groups treated with the folate receptor-targeted nanoemulsion formulation of the present disclosure significantly improved compared to that of the control and free docetaxel groups. Encapsulation of docetaxel in the nanoemulsion formulation sequesters it from normal tissue to reduce therapy related systemic toxicity, while still allowing the docetaxel to inhibit the division of cancer cells in tumors. The nanoemulsion formulations of the present disclosure, in which targeting ligands are present, are thus useful as anticancer delivery systems. The nanoemulsion formulations allowed for a more efficient chemotherapeutic delivery system, which had reduced systemic toxicity while still functioning to inhibit the division of cancer cells.

Encapsulation of docetaxel as part of the oil core of the nanoemulsion formulation according to the disclosure and inclusion of folate receptor-targeting modified lipids allowed for targeting moieties to be attached to an amphiphile of the interfacial surface membrane of the nanoemulsion formulation. This folate receptor-targeted nanoemulsion formulation created a more efficient chemotherapeutic delivery system that is effective against MDR cancers when compared to free docetaxel.

In order to determine if the nanoemulsion formulations according to the disclosure produce had safety in vivo the following experiment was performed. Following the completion of efficacy study, blood samples were collected from Taxotere, non-targeted and folate receptor-targeted nanoemulsion treated mice and untreated control mice. Liver enzyme (alanine amino-transferase (ALT) and aspartate amino-transferase (AST)) levels in blood were measured to detect any drug-induced toxicity to the liver.

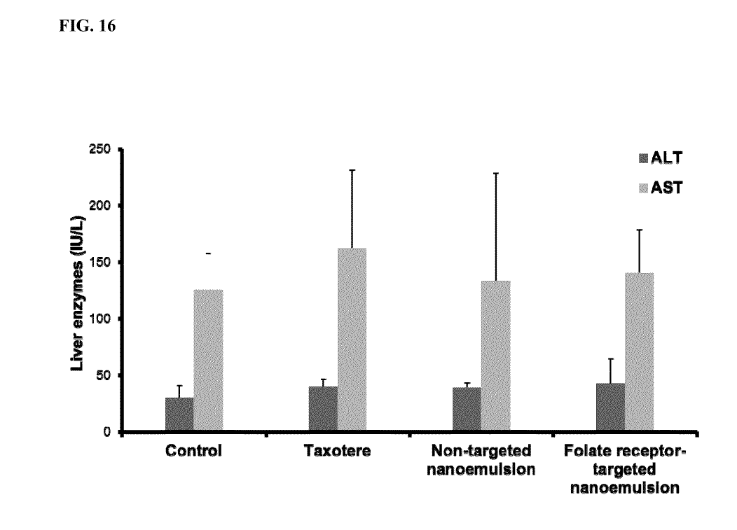

The results from FIG. 16 show that ALT & AST levels were slightly elevated in case of treatment groups as compared to control group, although not statistically significant, P>0.05. However, the important observation is that the non-targeted and folate receptor-targeted nanoemulsion formulation groups received 3 more doses than the Taxotere group, and still their AST & ALT levels were lower than the Taxotere group. These data indicate that the nanoemulsion formulations disclosed herein are safer in vivo than free docetaxel.

### Example 11

### In vivo MRI Studies Using Gadolinium-Labeled Nanoemulsion Formulations

That a gadolinium-based MRI contrasting agent is useful in the nanoemulsion formulations according to the disclosure was demonstrated as follows:

Three female Nu/Nu mice (Charles River Laboratories, Cambridge, Mass.) each weighing approximately 20 g and bearing subcutaneous SKOV3 tumors (human ovary cancer cells, American Type Culture Collection (ATCC), Manassas, Va.) approximately 200 mm to 300 mm in size, were used as test subjects. The first mouse was intravenously injected with gadopentetic acid containing a 0.072 mmol/kg dose of Gd-DTPA-PE (Magnevist™). The second mouse was intravenously injected with a non-targeted Gd-labeled nanoemulsion formulation of the present disclosure containing a 0.072 mmol/kg dose of Gd-DTPA-PE. The third mouse was intravenously injected with a folate receptor-targeted Gd-labeled nanoemulsion formulation of the present disclosure containing a 0.072 mmol/kg dose of the gadolinium-based MRI contrasting agent Gd-DTPA-PE. All three mice were full body scanned and imaged using a Bruker Biospec 20/70 MRI machine over a period of 24 hr.

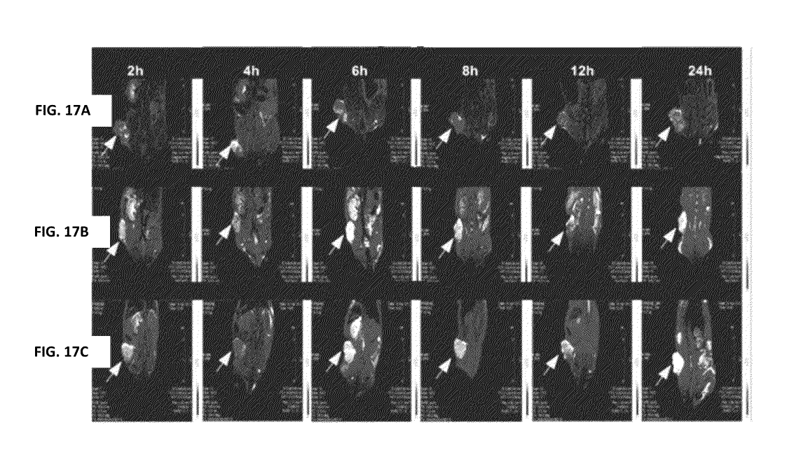

Representations of the resulting images are shown in FIGS. 17A-FIG. 17C. The images show preferential accumulation of the Gd-containing nanoemulsion formulations of the present disclosure in the subcutaneous flank SKOV3 tumors as compared to that of the contrasting agent control Magnevist™. The Magnevist™ control was observed at tumors between 2 hr to 4 hr as seen in FIG. 17A; whereas the Gd-labeled non-targeted nanoemulsion formulation of the present disclosure seen in FIG. 17B shows contrast enhancement of tumors between 6 hr to 24 hr. The Gd-labeled folate-targeted nanoemulsion formulations of the present disclosure seen in FIG. 17C improved contrast enhancement of tumors between 6 hr to 24 hr when compared to controls. The Magnevist™ control rapidly accumulated in the tumor over the first hr, but then cleared and resolved to near baseline by the 6th hr. In contrast, the nanoemulsion formulations of the present disclosure accumulated and remained in the tumor over a longer period of time, thereby enhancing tumor imaging.

These studies show that the Gd-labeled nanoemulsion formulations of the present disclosure are useful MRI agents, and are effective at imaging tumors over a longer period of time than the pure imaging agent Magnevist™.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A nanoemulsion formulation comprising:
an oil phase;
an interfacial surface membrane;
an aqueous phase;
a docetaxel compound dispersed in the oil phase; and
a targeting ligand comprising a folate receptor-targeting ligand.

2. The nanoemulsion formulation of claim 1, wherein the oil phase comprises flax seed oil, hemp oil, pumpkin seed oil, wheat germ oil, rice bran oil, canola oil, or any combination thereof.

3. The nanoemulsion formulation of claim 1, wherein the interfacial surface membrane phase comprises an emulsifier, a stabilizer, or any combination thereof.

4. The nanoemulsion formulation of claim 3, wherein the emulsifier in the interfacial surface membrane comprises egg lecithin, soy lecithin, phosphatidyl ethanolamine, phosphatidyl inositol, dimyristoylphosphatidyl choline, dimyristoylphosphatidyl ethyl-N-dimethyl propyl ammonium hydroxide nonionic or any combination thereof.

5. The nanoemulsion formulation of claim 3, wherein the stabilizer in the interfacial surface membrane comprises a polyethylene glycol derivative, a phosphatide, a polyglycerol mono oleate, or any combination thereof.

6. The nanoemulsion formulation of claim 1, wherein the docetaxel compound comprises docetaxel, lauroyl docetaxel, dilauroyl docetaxel, trilauroyl docetaxel or any combination thereof.

7. The nanoemulsion formulation of claim 1, wherein the folate receptor-targeting ligand comprises DSPE-PEG(2000)-cysteine-folic acid, DSPE-PEG(3400)-cysteine folic acid, DSPE-PEG(5000)-cysteine folic acid, an anti-folate receptor immunoglobulin or a folate receptor-binding fragment thereof, or any combination thereof.

8. The nanoemulsion formulation of claim 1, further comprising an imaging agent.

9. The nanoemulsion formulation of claim 8, wherein the imaging agent comprises a magnetic resonance imaging contrasting moiety.

10. The nanoemulsion formulation of claim 9, wherein the magnetic resonance imaging contrasting moiety comprises gadolinium, iron oxide, iron, platinum, manganese, or any combination thereof.

11. A method of imaging a cancer in a patient, comprising administering to a patient an amount of the nanoemulsion formulation of claim 8 sufficient to image the cancer.

12. A method of inhibiting or killing cancer cells, comprising a contacting the cells with an amount of the nanoemulsion formulation of claim 1 that is toxic to, or which inhibits the growth of, the cells.

13. The method of claim 12, wherein the cancer cells are in a mammal and the contacting step comprises administering to a mammal a therapeutically effective amount of the nanoemulsion formulation of claim 1.
